SEQUENCE LISTING (127 sequences)

SEQ ID NO: 1 - DNA, 8050 bp, Homo sapiens
ccataagaaactttttaaaaataaaaaaaactataataaaaattataagacactgtaaatgaatggttttagtcatctgaattttctctgacccttagaattaaggaaaaaaaaaacacacaatagttatggccaataataattcctccttctttgaggaaattcaacagttttagaaccaactttaaactatacaacctggcatatacacatttttaaaaacacacacacaaaattgtttactaagcaagaaggagaattcattaaagagccacgccagctacaggaagtttagattttaaatgtaaagagaggaggcaatggtacctgacttttcacattttgatttatattggcttctttccagaaaacgctttcactggctttcaaaaaatttttattttatctccaacccttagacggaaaccatcttcaactggaaatcagaacacctaggttttactttttatttaggcaaaaatgtatttggagccaggcatggtggatcacaactgtaatcccagcactttgggaggccaaggtgggcagataatctgtggtcaggagttcgagatgagcctggccaacatggcaaaaccccatctctactaaaaatataaaaattagccaggtgtggtcttgcatgcctgtggtctcaggtacttggaaggctgaggcaggagaatcacttaagcccaggaaacggaggttgcagtgagccaagatcacaccattgcactgggcgacagagcgagactccatcaaaaataataataatgaaataaataaataaataaaaagcacatttggtctccacctactggaaaacaggcaggaattgctgtgtgtttcacagacatggaccacgaagaggataaagtgagttaacttgttaagtgctaagaaatattctccataaacccaagctattatagttacgtaatatgagtctcttctctggaggcggtcttcatggattgtttgccagcctagccttaaaaatgtcaggcatcgaagatggccgaatagcaacagctcccgtctacagctcccagcgtgagcgacgcagaagacgggtgatttctgcatttccatctgaggtaccgggttcatctcactagggagtgccagacagtgggcgcaggccagtgtgtgtgcgcaccgtgtgccagccgaagcagggcgaggcattgcctcacctgggaagcgcaaggggtcagggagttccctttccgagtcaaagaaaggggtgaccgacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagccaggaagctcgaactgggtggagcccaccacagctcaaggaggcctgcctgcctctgtaggctccacctctgggggcagggcacagacaaacaaaaagacagcagtaacctctgcagacttaaatgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacacagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcaagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggacaaataaaatactttatagacaagcaaatgctgagagattttgtcaccaccaggcctgccctaaaagagctcctgaaggaagcgctaaacatggaaaggaacaatcggtaccagccgctgcaaaatcatgccaaaatgtaaagaccatcaagactaggaagaaactgcatcaactaatgagcaaaatcaccagctaacatcataatgacaggatcaaattcacacataacaatattaactttaaatgtaaatggactaaattctgcaattaaaagacacagactggcaagttggataaagagtcaagacccatcagtgtgctgtattcaggaaacccatctcgcgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacgagaggagctaactatcctaaatatttatgcacccaatacaggagcacccagattcataaagcaagtcctgagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcccacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagagatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacatggaggaactggtaccattccttctgaaactattccaatcaatagaaaaagagggaatcctccctaactcattttatgaggccagcatcattctgataccaaagccgggcagagacacaaccaaaaaagagaattttagaacaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggatgtatttcaaaataataagagctatctatgacaaacccacagccaatatcattctgaatgggcaaaaaccggaaacattccctttgaaaattggcacaagacagggatgccctctctcaccgctcctattcaacatagtgttggaagttctggccagggcaatcaggcaggagaaggaaataaagggtattcaattagaaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcatggtactggtaccaaaacagagatatagagcaatggaacagaacagagccctcagaaataatgccacatatctacaactatctgagctttgacaaacctgagaaaaacaagcaatggggaaaggattccctatttaataaatggtgctgggaaaactggctggccatatgtagaaagctgaaactggatcccttccttacaccttatacaaaaattaattcaagatggattaaagatttaaacgttagacctaaaaccataaaaaccctagaagaaaacctaggcattaccattcaggacataggcgtgggcaaggacttcatgtctaaaacaccaaaagcaatggcaacaaaagccaaaattgacaaatgggatctaattaaactaaagagctcctgcacagcaaaagaaactaccatcagagagaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaaaaaatgctcaccatcactggccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacatgtatgtttattgcggcactattcacaatagcaaggacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcacaagaacaaaaaaccaaacaccgcatattctcactcataggtgggaattgaacaatgagatcacttggacacaggaaggggaatatcacactctggggactgttgtggggtggggggaggggggagggatagcatgggagatatacctaatgctagatgacacgttagtgggtgcagcgcaccagcatggcacatgtatacatatgtaactaacctgcacaatgtgcacatgtaccctaaaacttagagtataataaaaaaaaaaaaatttaaaaaaaaaaaaaaaatgcaagtttagttaacattttcagccagcatgctaaaccttgtgatgaagtcatagggtcttatactcacgatttgataacataatagaactattaaaagaagtctgattcattaaaaaaaaaaaaaaatgtcaggcatcacaaacagggaaacctataaatgagaaatttggctgcagtcagtcccaggggactctctggcactggtgctgtggggactcacagtttaatcagttaaatctgcatcaggaagtcagctccccttctgctgcacccgctggtgtcacagaggtcctgagcatcaagccaacacctcctgctgagtagcaaaacgcttgtgccacctgccatcctggggaaaacagccatgctgctggagagtgtaatcaatggagagagagcccggtcacttctgccatcttgctctcctcccacctgggaatagaagtgacaactattgagccatcagaatgaaaatctttactagccctgcagtgatccaggtacatagagtgacctgcaacactacagttttaggaaggatattgattgtcttatctaacaggaaaatgtgaacccagtttatattttaggttaactctgtctcaggctttctcactttggttctttcacacatattcaacaaacatccattgtctgtaatgttccaggcactgttctaggtcatatgaaaaatcagaattgaacagacatagccacttactctcaaagagtttactattcagtaaaaggaaagaagataagtgcaatataaagttataatgatacaagacaggtgtgtctatttgcatatatatttatataaaaatgaataaaatcttgaggctatttttatttaataacgtctcaggatactataagaggtgaggagaagggccaatggattgagattcactaatgactcaaggtatgataattactattctctgttgtttttaaaaaaacttttcttcatgtaatgatctacgggcattttt

SEQ ID NO: 2 - DNA, 25 bp, Artificial Sequence
  Synthetic sequence of forward primer for L1 5′UTR-ORF1
gaatgattttgacgagctgagagaa

SEQ ID NO: 3 - DNA, 24 bp, Artificial Sequence
  Synthetic sequence of reverse primer for L1 5′ UTR-ORF1
gtcctcccgtagctcagagtaatt

SEQ ID NO: 4 - DNA, 16 bp, Artificial Sequence
  Synthetic probe sequence for L1 5′ UTR-ORF1
aaggcttcagacgatc

SEQ ID NO: 5 - DNA, 23 bp, Artificial Sequence
  Synthetic sequence of forward primer for L1 ORF2
tgcggagaaataggaacactttt

SEQ ID NO: 6 - DNA, 21 bp, Artificial Sequence
  Synthetic sequence of reverse primer for L1 ORF2
tgaggaatcgccacactgact

SEQ ID NO: 7 - DNA, 21 bp, Artificial Sequence
  Synthetic probe sequence for L1 ORF2
ctgtaaactagttcaaccatt

SEQ ID NO: 8 - DNA, 22 bp, Artificial Sequence
  Synthetic sequence of forward primer for SATA
ggtcaatggcagaaaaggaaat

SEQ ID NO: 9 - DNA, 21 bp, Artificial Sequence
  Synthetic sequence of reverse primer for SATA
cgcagtttgtgggaatgattc

SEQ ID NO: 10 - DNA, 18 bp, Artificial Sequence
  Synthetic probe sequence for SATA
tcttcgtttcaaaactag

SEQ ID NO: 11 - DNA, 20 bp, Artificial Sequence
  Synthetic sequence of forward primer for RPL13A
gaaagccaagatccactacc

SEQ ID NO: 12 - DNA, 20 bp, Artificial Sequence
  Synthetic sequence of reverse primer for RPL13A
tgggtcttgaggacctctgt

SEQ ID NO: 13 - DNA, 22 bp, Artificial Sequence
  Synthetic sequence of forward primer for RUNX2
tcaacgatctgagatttgtggg

SEQ ID NO: 14 - DNA, 21 bp, Artificial Sequence
  Synthetic sequence of reverse primer for RUNX2
ggggaggatttgtgaagacgg

SEQ ID NO: 15 - DNA, 20 bp, Artificial Sequence
  Synthetic sequence of forward primer for OCN
ggcgctacctgtatcaatgg

SEQ ID NO: 16 - DNA, 19 bp, Artificial Sequence
  Synthetic sequence of reverse primer for OCN
gtggtcagccaactcgtca

SEQ ID NO: 17 - DNA, 21 bp, Artificial Sequence
  Synthetic sequence of forward primer for OPN
gaagtttcgcagacctgacat

SEQ ID NO: 18 - DNA, 22 bp, Artificial Sequence
  Synthetic sequence of reverse primer for OPN
gtatgcaccattcaactcctcg

SEQ ID NO: 19 - DNA, 21 bp, Artificial Sequence
  Synthetic sequence of forward primer for BSP
cactggagccaatgcagaaga

SEQ ID NO: 20 - DNA, 21 bp, Artificial Sequence
  Synthetic sequence of reverse primer for BSP
tggtggggttgtaggttcaaa

SEQ ID NO: 21 - DNA, 20 bp, Artificial Sequence
  Synthetic sequence of forward primer for OSX
cctctgcgggactcaacaac

SEQ ID NO: 22 - DNA, 22 bp, Artificial Sequence
  Synthetic sequence of reverse primer for OSX
agcccattagtgcttgtaaagg

SEQ ID NO: 23 - DNA, 20 bp, Artificial Sequence
  Synthetic sequence of forward primer for TBP
gctggcccatagtgatcttt

SEQ ID NO: 24 - DNA, 21 bp, Artificial Sequence
  Synthetic sequence of reverse primer for TBP
cttcacacgccaagaaacagt

SEQ ID NO: 25 - DNA, 22 bp, Artificial Sequence
  Synthetic sequence of forward primer for PPARgamma
accaaagtgcaatcaaagtgga

SEQ ID NO: 26 - DNA, 19 bp, Artificial Sequence
  Synthetic sequence of reverse primer for PPARgamma
ctcgtggaagtgacgcctt

SEQ ID NO: 27 - DNA, 20 bp, Artificial Sequence
  Synthetic sequence of forward primer for FABP4
actgggccaggaatttgacg

SEQ ID NO: 28 - DNA, 19 bp, Artificial Sequence
  Synthetic sequence of reverse primer for FABP4
ctcgtggaagtgacgcctt

SEQ ID NO: 29 - DNA, 23 bp, Artificial Sequence
  Synthetic sequence of forward primer for FASN
aaggacctgtctaggtttgatgc

SEQ ID NO: 30 - DNA, 21 bp, Artificial Sequence
  Synthetic sequence of reverse primer for FASN
tggcttcataggtgacttcca

SEQ ID NO: 31 - DNA, 20 bp, Artificial Sequence
  Synthetic sequence of forward primer for LPL
aggatgtggcccggtttatc

SEQ ID NO: 32 - DNA, 22 bp, Artificial Sequence
  Synthetic sequence of reverse primer for LPL
ccaaggctgtatcccaagagat

SEQ ID NO: 33 - DNA, 23 bp, Artificial Sequence
  Synthetic sequence of forward primer for GFP-968/1013
gcaccatcttcttcaaggacgac

SEQ ID NO: 34 - DNA, 20 bp, Artificial Sequence
  Synthetic sequence of reverse primer for GFP-968/1013
tctttgctcagggcggactg

SEQ ID NO: 35 - DNA, 20 bp, Artificial Sequence
  Synthetic sequence of forward primer for mp16
cgtgaacatgttgttgaggc

SEQ ID NO: 36 - DNA, 20 bp, Artificial Sequence
  Synthetic sequence of reverse primer for mp16
gcagaagagctgctacgtga

SEQ ID NO: 37 - DNA, 20 bp, Artificial Sequence
  Synthetic sequence of forward primer for mp21
cggtgtcagagtctagggga

SEQ ID NO: 38 - DNA, 20 bp, Artificial Sequence
  Synthetic sequence of reverse primer for mp21
atcaccaggattggacatgg

SEQ ID NO: 39 - DNA, 18 bp, Artificial Sequence
  Synthetic sequence of forward primer for mAtf3
ctctggccgttctctgga

SEQ ID NO: 40 - DNA, 20 bp, Artificial Sequence
  Synthetic sequence of reverse primer for mAtf3
ggtcgcactgacttctgagg

SEQ ID NO: 41 - DNA, 19 bp, Artificial Sequence
  Synthetic sequence of forward primer for mGadd45b
cggccaaactgatgaatgt

SEQ ID NO: 42 - DNA, 20 bp, Artificial Sequence
  Synthetic sequence of reverse primer for mGadd45b
tctgcagagcgatatcatcc

SEQ ID NO: 43 - DNA, 20 bp, Artificial Sequence
  Synthetic sequence of forward primer for mBtg2
gcgagcagagactcaaggtt

SEQ ID NO: 44 - DNA, 20 bp, Artificial Sequence
  Synthetic sequence of reverse primer for mBtg2
tagccagaacctttggatgg

SEQ ID NO: 45 - DNA, 20 bp, Artificial Sequence
  Synthetic sequence of forward primer for mMMP13
tgatgaaacctggacaagca

SEQ ID NO: 46 - DNA, 20 bp, Artificial Sequence
  Synthetic sequence of reverse primer for mMMP13
ggtccttggagtgatccaga

SEQ ID NO: 47 - DNA, 20 bp, Artificial Sequence
  Synthetic sequence of forward primer for mIL6
tgatgcacttgcagaaaaca

SEQ ID NO: 48 - DNA, 23 bp, Artificial Sequence
  Synthetic sequence of reverse primer for mIL6
accagaggaaattttcaataggc

SEQ ID NO: 49 - DNA, 18 bp, Artificial Sequence
  Synthetic sequence of forward primer for mLap2a
ttctcgagcgacgaggag

SEQ ID NO: 50 - DNA, 20 bp, Artificial Sequence
  Synthetic sequence of reverse primer for mLap2a
agcctgggcttatcagtttt

SEQ ID NO: 51 - DNA, 20 bp, Artificial Sequence
  Synthetic sequence of forward primer for mGapdh
ggcaaattcaacggcacagt

SEQ ID NO: 52 - DNA, 20 bp, Artificial Sequence
  Synthetic sequence of reverse primer for mGapdh
gtctcgctcctggaagatgg

SEQ ID NO: 53 - DNA, 20 bp, Artificial Sequence
  Synthetic sequence of forward primer for mL1
gcggttcctcagaaaattgg

SEQ ID NO: 54 - DNA, 20 bp, Artificial Sequence
  Synthetic sequence of reverse primer for mL1
tgcccaggagaggtattgct

SEQ ID NO: 55 - protein, 11 aa, Artificial Sequence
  Synthetic sequence encoding peptide TD-1
ACSSSPSKHCG

SEQ ID NO: 56 - protein, 11 aa, Artificial Sequence
  Synthetic sequence encoding the SPACE peptide
ACTGSTQHQCG

SEQ ID NO: 57 - DNA, 21 bp, Artificial Sequence
  Synthetic sequence of oligo against L1 5′ UTR
gtacctcagatggaaatgcag

SEQ ID NO: 58 - DNA, 21 bp, Artificial Sequence
  Synthetic sequence of oligo against L1 5′ UTR
atgaacccggtacctcagatg

SEQ ID NO: 59 - DNA, 21 bp, Artificial Sequence
  Synthetic sequence of oligo against L1 5′ UTR
ccctagtgagatgaacccggt

SEQ ID NO: 60 - DNA, 21 bp, Artificial Sequence
  Synthetic sequence of oligo against L1 5′ UTR
gtctggcactccctagtgaga

SEQ ID NO: 61 - DNA, 21 bp, Artificial Sequence
  Synthetic sequence of oligo against L1 5′ UTR
tgcgcccactgtctggcactc

SEQ ID NO: 62 - DNA, 21 bp, Artificial Sequence
  Synthetic sequence of oligo against L1 5′ UTR
cacactggcctgcgcccactg

SEQ ID NO: 63 - DNA, 21 bp, Artificial Sequence
  Synthetic sequence of oligo against L1 5′ UTR
ggtgcgcacacacactggcct

SEQ ID NO: 64 - DNA, 21 bp, Artificial Sequence
  Synthetic sequence of oligo against L1 5′ UTR
gctcgcgcaaggtgcgcacac

SEQ ID NO: 65 - DNA, 21 bp, Artificial Sequence
  Synthetic sequence of oligo against L1 5′ UTR
ccctgcttcggctcgcgcaag

SEQ ID NO: 66 - DNA, 21 bp, Artificial Sequence
  Synthetic sequence of oligo against L1 5′ UTR
caatgcctcgccctgcttcgg

SEQ ID NO: 67 - DNA, 21 bp, Artificial Sequence
  Synthetic sequence of oligo against L1 5′ UTR
ccaggtgaggcaatgcctcgc

SEQ ID NO: 68 - DNA, 21 bp, Artificial Sequence
  Synthetic sequence of oligo against L1 5′ UTR
cttgcgcttcccaggtgaggc

SEQ ID NO: 69 - DNA, 21 bp, Artificial Sequence
  Synthetic sequence of oligo against L1 5′ UTR
tccctgaccccttgcgcttcc

SEQ ID NO: 70 - DNA, 21 bp, Artificial Sequence
  Synthetic sequence of oligo against L1 5′ UTR
gaaagggaactccctgacccc

SEQ ID NO: 71 - DNA, 21 bp, Artificial Sequence
  Synthetic sequence of oligo against L1 5′ UTR
ctttgactcggaaagggaact

SEQ ID NO: 72 - DNA, 21 bp, Artificial Sequence
  Synthetic sequence of oligo against L1 5′ UTR
tcacccctttctttgactcgg

SEQ ID NO: 73 - DNA, 21 bp, Artificial Sequence
  Synthetic sequence of oligo against L1 5′ UTR
ggtgcgtccgtcacccctttc

SEQ ID NO: 74 - DNA, 21 bp, Artificial Sequence
  Synthetic sequence of oligo against L1 5′ UTR
cgattttccaggtgcgtccgt

SEQ ID NO: 75 - DNA, 21 bp, Artificial Sequence
  Synthetic sequence of oligo against L1 5′UTR
gggagtgacccgattttccag

SEQ ID NO: 76 - DNA, 21 bp, Artificial Sequence
  Synthetic sequence of oligo against L1 5′ UTR
atattcgggtgggagtgaccc

SEQ ID NO: 77 - DNA, 21 bp, Artificial Sequence
  Synthetic sequence of oligo against L1 ORF1
ctttgttctgttgctggtgag

SEQ ID NO: 78 - DNA, 21 bp, Artificial Sequence
  Synthetic sequence of oligo against L1 ORF1
ctccatccagctttgttctgt

SEQ ID NO: 79 - DNA, 21 bp, Artificial Sequence
  Synthetic sequence of oligo against L1 ORF1
caaaatcattctccatccagc

SEQ ID NO: 80 - DNA, 21 bp, Artificial Sequence
  Synthetic sequence of oligo against L1 ORF1
ctcagctcgtcaaaatcattc

SEQ ID NO: 81 - DNA, 21 bp, Artificial Sequence
  Synthetic sequence of oligo against L1 ORF1
gccttcttctctcagctcgtc

SEQ ID NO: 82 - DNA, 21 bp, Artificial Sequence
  Synthetic sequence of oligo against L1 ORF1
atcgtctgaagccttcttctc

SEQ ID NO: 83 - DNA, 21 bp, Artificial Sequence
  Synthetic sequence of oligo against L1 ORF1
gagtaatttgatcgtctgaag

SEQ ID NO: 84 - DNA, 21 bp, Artificial Sequence
  Synthetic sequence of oligo against L1 ORF1
ccgtagctcagagtaatttga

SEQ ID NO: 85 - DNA, 21 bp, Artificial Sequence
  Synthetic sequence of oligo against L1 ORF1
gaatgtcctcccgtagctcag

SEQ ID NO: 86 - DNA, 21 bp, Artificial Sequence
  Synthetic sequence of oligo against L1 ORF1
cctttggtttgaatgtcctcc

SEQ ID NO: 87 - DNA, 21 bp, Artificial Sequence
  Synthetic sequence of oligo against L1 ORF1
aacttctttgcctttggtttg

SEQ ID NO: 88 - DNA, 21 bp, Artificial Sequence
  Synthetic sequence of oligo against L1 ORF1
caaagttttgaacttctttgc

SEQ ID NO: 89 - DNA, 21 bp, Artificial Sequence
  Synthetic sequence of oligo against L1 ORF1
aaatttttttcaaagttttga

SEQ ID NO: 90 - DNA, 21 bp, Artificial Sequence
  Synthetic sequence of oligo against L1 ORF1
acattcttctaaatttttttc

SEQ ID NO: 91 - DNA, 21 bp, Artificial Sequence
  Synthetic sequence of oligo against L1 ORF1
ttctagttatacattcttcta

SEQ ID NO: 92 - DNA, 21 bp, Artificial Sequence
  Synthetic sequence of oligo against L1 ORF1
gtattggttattctagttata

SEQ ID NO: 93 - DNA, 21 bp, Artificial Sequence
  Synthetic sequence of oligo against L1 ORF1
gcacttctctgtattggttat

SEQ ID NO: 94 - DNA, 21 bp, Artificial Sequence
  Synthetic sequence of oligo against L1 ORF1
gctcctttaagcacttctctg

SEQ ID NO: 95 - DNA, 21 bp, Artificial Sequence
  Synthetic sequence of oligo against L1 ORF1
agctccatcagctcctttaag

SEQ ID NO: 96 - DNA, 21 bp, Artificial Sequence
  Synthetic sequence of oligo against L1 ORF1
cttggttttcagctccatcag

SEQ ID NO: 97 - DNA, 21 bp, Artificial Sequence
  Synthetic sequence of oligo against L1 ORF2
tgttatgtgtgaatttgatcc

SEQ ID NO: 98 - DNA, 21 bp, Artificial Sequence
  Synthetic sequence of oligo against L1 ORF2
aagttaatattgttatgtgtg

SEQ ID NO: 99 - DNA, 21 bp, Artificial Sequence
  Synthetic sequence of oligo against L1 ORF2
tttatatttaaagttaatatt

SEQ ID NO: 100 - DNA, 21 bp, Artificial Sequence
  Synthetic sequence of oligo against L1 ORF2
atttagtccatttatatttaa

SEQ ID NO: 101 - DNA, 21 bp, Artificial Sequence
  Synthetic sequence of oligo against L1 ORF2
taattgcagaatttagtccat

SEQ ID NO: 102 - DNA, 21 bp, Artificial Sequence
  Synthetic sequence of oligo against L1 ORF2
ctgtgtcttttaattgcagaa

SEQ ID NO: 103 - DNA, 21 bp, Artificial Sequence
  Synthetic sequence of oligo against L1 ORF2
acttgccagtctgtgtctttt

SEQ ID NO: 104 - DNA, 21 bp, Artificial Sequence
  Synthetic sequence of oligo against L1 ORF2
tctttatgcaacttgccagtc

SEQ ID NO: 105 - DNA, 21 bp, Artificial Sequence
  Synthetic sequence of oligo against L1 ORF2
gggtcttgactctttatgcaa

SEQ ID NO: 106 - DNA, 21 bp, Artificial Sequence
  Synthetic sequence of oligo against L1 ORF2
gcacactgatgggtcttgact

SEQ ID NO: 107 - DNA, 21 bp, Artificial Sequence
  Synthetic sequence of oligo against L1 ORF2
tgtgggatcggtggtgatatc

SEQ ID NO: 108 - DNA, 21 bp, Artificial Sequence
  Synthetic sequence of oligo against L1 ORF2
tttgtatttctgtgggatcgg

SEQ ID NO: 109 - DNA, 21 bp, Artificial Sequence
  Synthetic sequence of oligo against L1 ORF2
ctgatggtagtttgtatttct

SEQ ID NO: 110 - DNA, 21 bp, Artificial Sequence
  Synthetic sequence of oligo against L1 ORF2
gtagtattctctgatggtagt

SEQ ID NO: 111 - DNA, 21 bp, Artificial Sequence
  Synthetic sequence of oligo against L1 ORF2
agaggtgtttgtagtattctc

SEQ ID NO: 112 - DNA, 21 bp, Artificial Sequence
  Synthetic sequence of oligo against L1 ORF2
ttatttgcgtagaggtgtttg

SEQ ID NO: 113 - DNA, 21 bp, Artificial Sequence
  Synthetic sequence of oligo against L1 ORF2
attttctactttatttgcgta

SEQ ID NO: 114 - DNA, 21 bp, Artificial Sequence
  Synthetic sequence of oligo against L1 ORF2
tttcttctagattttctactt

SEQ ID NO: 115 - DNA, 21 bp, Artificial Sequence
  Synthetic sequence of oligo against L1 ORF2
aatgtatccatttcttctaga

SEQ ID NO: 116 - DNA, 21 bp, Artificial Sequence
  Synthetic sequence of oligo against L1 ORF2
tgtgtcgaggaatgtatccat

SEQ ID NO: 117 - DNA, 21 bp, Artificial Sequence
  Synthetic sequence of oligo against L1 3′ UTR
tagcattaggtatatctccca

SEQ ID NO: 118 - DNA, 21 bp, Artificial Sequence
  Synthetic sequence of oligo against L1 3′ UTR
atgtgtcatctagcattaggt

SEQ ID NO: 119 - DNA, 21 bp, Artificial Sequence
  Synthetic sequence of oligo against L1 3′ UTR
gcacccactaatgtgtcatct

SEQ ID NO: 120 - DNA, 21 bp, Artificial Sequence
  Synthetic sequence of oligo against L1 3′ UTR
ctggtgcgctgcacccactaa

SEQ ID NO: 121 - DNA, 21 bp, Artificial Sequence
  Synthetic sequence of oligo against L1 3′ UTR
atgtgccatgctggtgcgctg

SEQ ID NO: 122 - DNA, 21 bp, Artificial Sequence
  Synthetic sequence of oligo against L1 3′ UTR
atatgtatacatgtgccatgc

SEQ ID NO: 123 - DNA, 21 bp, Artificial Sequence
  Synthetic sequence of oligo against L1 3′ UTR
ggttagttacatatgtataca

SEQ ID NO: 124 - DNA, 21 bp, Artificial Sequence
  Synthetic sequence of oligo against L1 3′ UTR
acattgtgcaggttagttaca

SEQ ID NO: 125 - DNA, 21 bp, Artificial Sequence
  Synthetic sequence of oligo against L1 3′ UTR
gtacatgtgcacattgtgcag

SEQ ID NO: 126 - DNA, 21 bp, Artificial Sequence
  Synthetic sequence of oligo against L1 3′ UTR
aagttttagggtacatgtgca

SEQ ID NO: 127 - DNA, 21 bp, Artificial Sequence
  Synthetic sequence of oligo against L1 3′ UTR
attatactctaagttttaggg

(19) United States
(12) Patent Application Publication
(10) Pub. No.: US 2025/0290065 A1
(43) Pub. Date: Sep. 18, 2025

(54) METHODS FOR MODULATING HUMAN L1 RETROTRANSPOSONS RNA AND COMPOSITIONS FOR USE THEREIN

(71) Applicants: King Abdullah University of Science and Technology, Thuwal (SA); Salk Institute For Biological Studies, La Jolla, CA (US)

(72) Inventors: Valerio Orlando, Thuwal (SA); Francesco Della Valle, Thuwal (SA); Arianna Mangiavacchi, Thuwal (SA); Juan Carlos Izpisua-Belmonte, La Jolla, CA (US); Pradeep Dubbaka Venu Reddy, La Jolla, CA (US)

(21) Appl. No.: 17/769,524

(22) PCT Filed: Oct. 16, 2020
(86) PCT No.: PCT/US2020/056097

§ 371 Date: Apr. 15, 2022

Related U.S. Application Data
(60) Provisional application No. 62/916,096, filed on Oct. 16, 2019.
(60) Provisional application No. 62/945,535, filed on Dec. 9, 2019.

(51) Int. Cl. C12N 15/113

(52) U.S. Cl. CPC C12N 15/113; C12N 2310/113; C12N 2320/32

(57) ABSTRACT

Compositions and methods for upregulating L1 RNA activity in a subject in need thereof are provided. The compositions include nucleic acids encoding L1 RNA or the L1 RNA, alone, or contained in an expression vector and/or further contained within osteogenic progenitor cells, for example, mesenchymal stem cells, genetically engineering to express L1 RNA. In this aspect, the compositions are used to increase L1 RNA levels for example, L1 RNA copy number in subjects in need of increasing their bone mass index. In a preferred embodiment, the bone progenitor cells are autologous cells.

Compositions and methods for downregulating L1 RNA levels/activity in a subject in need thereof are also provided. The compositions include one or more agents in effective amounts to knockdown L1 RNA in a cell. The compositions can be used to treat conditions associated with ageing. A preferred agent is a L1 RNA antisense oligonucleotide.

### CROSS REFERENCE TO RELATED APPLICATIONS

The present application is a National Phase application under 35 U.S.C. 371 of PCT/US2020/056097, filed Oct. 16, 2020, which claims priority to and benefit of U.S. Application No. 62/916,096, filed Oct. 16, 2019, and U.S. Application No. 62/945,535, filed Dec. 9, 2019, the disclosures of which are incorporated herein by reference in their entirety.

### FIELD OF THE INVENTION

The invention is generally directed to methods for modulating human L1 retrotransposon RNA activity in a subject in need thereof, and compositions for use therein.

### REFERENCE TO SEQUENCE LISTING

The Sequence Listing submitted as a file named “SALK_2020_001A_371_ST25.txt” created on Mar. 28, 2024, and has a file size of 38,772 bytes is hereby incorporated by reference pursuant to 37 C.F.R. § 1.823 (b)(1).

### BACKGROUND OF THE INVENTION

Long interspersed nuclear elements (LINEs) are a group of non-LTR (long terminal repeat) retrotransposons which are widespread in the genome of many eukaryotes. LINEs make up a family of transposons, where each LINE is about 7000 base pairs long. LINEs are transcribed into mRNA and translated into protein that acts as a reverse transcriptase. The reverse transcriptase makes a DNA copy of the LINE RNA that can be integrated into the genome at a new site. The only abundant LINE in humans is LINE-1. L1 account for about 21% of the human genome (Lander, et al. Nature (2001), doi: 10.1038/35057062), but only a few tens, belonging to the L1HS (L1 human specific) Ta (Transcribed, subset a) subfamily, still retain the ability to retrotranspose autonomously (Sassaman, et al. Nat. Genet. (1997), doi: 10.1038/ng0597-37; Brouha, et al. Proc. Natl. Acad. Sci. (2003), doi: 10.1073/pnas.0831042100) through an ORF2-dependent RNA-mediated “copy and paste” mechanism (Fent, et al. Cell (1996), doi: 10.1016/S0092-8674 (00) 81997-2; Luan, et al., Cell (1993), doi: 10.1016/0092-8674 (93) 90078-5; Cost, et al. EMBO J. (2002), doi: 10.1093/emboj/cdf592.). Although the cells have evolved several defense mechanisms to prevent deleterious uncontrolled transposition (Kazazian, et al. N. Engl. J. Med. (2017), doi: 10.1056/NEJMra1510092), evidence indicates that somatic L1 mobilization occurs in developing brain, contributing to individual somatic mosaicism (Coufal, et al. Nature (2009), doi: 10.1038/nature08248; Muotri, et al. Hippocampus (2009), doi: 10.1002/hipo.20564; Baillie, et al. Nature (2011), doi: 10.1038/nature10531; Evrony, et al. Cell (2012), doi: 10.1016/j.cell.2012.09.035), although its function remains unknown. Interestingly, in mice, L1 reactivation in the brain correlates with exposure to early life stress conditions (Bedrosian, et al. Science 359 (6382): 1395-1399 (2018), doi: 10.1126/science.aah3378). However, whether L1 mobilization is supported by other tissues, and if L1 expansion may contribute to tissue homeostasis is largely unexplored.

It is an object of the present invention to provide compositions and methods for modulating L1 in a subject in need thereof.

### SUMMARY OF THE INVENTION

One embodiment provides compositions and methods for upregulating L1 RNA activity in a subject in need thereof. The L1 is preferably of the L1HS-Ta1 family. The compositions include nucleic acids encoding L1 RNA or the L1 RNA, alone, or contained in an expression vector. The NA is preferably in a pharmaceutically acceptable carrier to the subject, or it can be incorporated into bone marrow derived osteogenic progenitor cells, for example, mesenchymal stem cells, by genetically engineering the progenitor cells to express L1 RNA, and suspending the L1 RNA-expressing cells in a pharmaceutically acceptable carrier. In this aspect, the compositions are used to increase L1 RNA levels for example, L1 RNA copy number in subjects in need of increasing their bone mass index. Exemplary subjects include post-menopausal women, subjects diagnosed with osteoporosis or at risk of developing osteoporosis, and subjects on retroviral therapy, for example, NRT1. The methods include administering nucleic acids (NA) encoding L1 RNA or L1 RNA to the subject in need thereof. The NA can be administered in a pharmaceutically acceptable carrier to the subject, or it can be administered in the form of bone marrow derived osteogenic progenitor cells, for example, mesenchymal stem cells, genetically engineered to express L1 RNA, in a pharmaceutically acceptable carrier. In a preferred embodiment, the bone progenitor cells are autologous cells.

Another embodiment provides compositions and methods for downregulating L1 RNA levels/activity in a subject in need thereof. A preferred agent is a L1 RNA antisense oligonucleotide, particularly preferred are fluoroarabinonucleic acids (FANA) modified antisense oligonucleotides. The compositions include formulations containing one or more agents for depleting L1 RNA. In a preferred embodiment, the method includes downregulating L1 RNA levels/activity in cells in a subject, for example, fibroblasts, preferably, skin fibroblasts. The method in preferred embodiments include administering one or more agents in effective amounts to knockdown L1 RNA in cells in a subject, for example, skin fibroblasts. The compositions can be used to treat conditions associated with ageing and accelerated ageing, including but not limited to progeria syndrome and wrinkles.

### BRIEF DESCRIPTION OF THE DRAWINGS

FIGS. 1A-1H show L1 DNA copy number in bone biopsies of CTR and OP groups correlated to clinical parameters related to skeletal metabolism and to other clinical indices. (FIG. 1A) PCR primers and probes positioning and CNV assay of L1-5′UTR-ORF1 (left panel, OP/CTR P=0.0003) and L1 ORF2 (right panel, OP/CTR P=0.0002) sequences in bone genome of osteoporotic (OP) and healthy (CTR) postmenopausal women. The significance between the mean values was determined by one tailed Student's t test. Correlation analysis between individual L1 5′UTR-ORF1 copy number and clinical parameters related (FIGS. 1B-D) or not related (FIGS. 1E-1H) to skeleton metabolism. Squares and circles identify healthy (CTR) and osteoporotic (OP) participants, respectively.

FIGS. 2A-2G show the correlation between L1 ORF2 copy number and clinical parameters related to skeletal metabolism and to other clinical indices in CTR and OP groups. Correlation analysis between individual L1 ORF2 copy number and clinical parameters related (FIGS. 2A-2C) or not related (FIGS. 2D-2G) to skeleton metabolism. Squares and circles identify healthy (CTR) and osteoporotic (OP) participants, respectively. FIG. 2H shows L1 DNA copy number in blood and bone biopsies of CTR and OP groups. PCR primers and probes positioning and CNV assay of L1-5′UTR-ORF1 (left panel) and ORF2 (right panel) sequences in bone and peripheral blood mononuclear cells (PBMC) genome of healthy (CTR, N=13) and osteoporotic (OP, N=9) postmenopausal women. The results are given as normalized values in relation to healthy bone. The significance between the mean values was determined by one tailed Student's t test, comparing CTR bone with the others.

FIGS. 3A-3B show RNA expression and genomic CNV of L1 in differentiating osteoblasts. FIG. 3A) Model system: ex vivo osteogenesis of human bone marrow-derived mesenchymal stem cells. FIG. 3B) PCR primers and probes positioning and timeline of L1 expression and L1 copy number variation during ex vivo osteogenesis. Results come from separate experiments performed on three different donors (N=3). The significance between mean values was determined by unpaired one tailed Student's t test. FIG. 3C shows quantitative mineralization analysis for all the donors tested. Donors with earlier onset of mineralization (left panel) compared to the others were not included in the study (right panel). FIG. 3D) RUNX2 (Runt-related transcription factor 2); OSX (Osterix, SP7); OCN (osteocalcin); OPN (Osteopontin); BSP (Bone sialoprotein). FIG. 3E shows results from cells electroporated with a plasmid that contains a retrotransposition-competent human L1 (RC-L1) and a retrotransposition indicator cassette in L1 3′UTR, consisting of a reversed enhanced green fluorescent protein (EGFP) interrupted by an intron in the same transcriptional orientation as the L1. The orientation of the cassette ensures that spliced EGFP sequence in cell genomic DNA only arise after a round of retrotransposition. *1243 nt is the expected PCR amplicon length of intron-containing EGFP DNA sequence (not retrotransposed); *342 nt is the expected PCR amplicon length of EGFP DNA sequence after splicing and retrotransposition.

FIG. 4A shows L1 RNA knock-down strategy: FANA-ASOs are delivered to cells, bind the complementary sequence in L1 RNA and trigger the RNaseH-mediated degradation of L1transcript. FIG. 4B. Ratio of osteogenic genes expression between anti-L1 FANA-ASOs and negative control (SCR). L1 Knock-down reduces the expression of OCN (−10%, p=0.047), RUNX2 (−23%, p<0.001), OSX (−43%, p=0.066), BSP (−44%, p=0.018), OPN (−40%, p=0.005). FIG. 4C. Timeline of CNV of L15′UTR-ORF1 (left) and L1 ORF2 (right) in Lamivudine 3TC treated (3TC) and control (DMSO) cells. The result comes from separate experiments performed on three different donors (N=3). FIG. 4D shows the ratio of osteogenic genes expression between Lamivudine 3TC treated (3TC) and control (DMSO) cells. Day 14: OPN (−18%, p=0.016), OSX (−60%, p=0.002) BSP (−34%, p=0.015). Day 21: RUNX2 (+32%, p<0.001), OPN (−23%, p=0.069), OSX (−50%, p=0.058), BSP (−60%, p=0.002). Right panel: Lamivudine treated (3TC) and control (DMSO) cells after 14 and 21 days of differentiation. FIG. 4E, Left panel, timeline quantification of mineral deposition in Lamivudine 3TC treated (3TC) and control (DMSO) cells. Mineralization is reduced (−69%, p=0.003) in Lamivudine 3TC treated cells after 21 days of differentiation. Quantification of intracellular lipid content is used as negative control. The significance between mean values was determined by unpaired one tailed Student's t test. FIG. 4F shows knock-down efficiency of ASOs is 45% (left, p<0.001) for 5′UTR-ORF1 containing L1 sequences and 30% (right, p=0.003) for ORF2 containing L1 sequences. The significance between mean values was determined by unpaired one tailed Student's t test.

FIGS. 5A-5D show L1 dynamics and Lamivudine 3TC-mediated inhibition of L1 expansion in differentiating adipocytes. FIG. 5A. Model system: ex vivo adipogenesis of bone marrow derived mesenchymal stem cells. FIG. 5B. PCR primers and probes positioning and timeline of L1 expression and L1 copy number variation during ex vivo adipogenesis. Results come from separate experiments performed on three different donors (N=3). FIG. 5C. Ratio of adipogenic genes expression between Lamivudine 3TC treated (3TC) and control (DMSO) cells. FIG. 5D shows timeline quantification of intracellular lipid content in Lamivudine 3TC treated (3TC) and control (DMSO) cells.

FIG. 6A-G show a correlation between L1 copy number and bone marker transcript signal levels in the cohort of 30 healthy and osteoporotic women. Squares and circles identify healthy and osteoporotic participants, respectively. SATA=human centromeric alpha satellite repeated DNA. The primary data for Affymetrix signal levels are available from the European Bioinformatics Institute (EMBL-EBI: ID: E-MEXP-1618).

FIG. 7A shows quantification of MSC mineralization after 14, 17 and 21 days of ex vivo differentiation. MSC were obtained from the femur of 4 healthy (D188, D239, D247, D170) and 4 OP patients (HUK7, HUK9, HUK12, HUK16). N=9 technical replicates for each donor and time point. FIG. 7B shows experimental workflow and flow cytometer analysis showing the percentage of positive cells 6 hours after L1 RNA delivery at day 7 of ex vivo osteogenesis. Intracellular localization of synthetic L1 (spots left of the broken line) and bone matrix (spots right of the broken line) production three days after transfection are also shown from a typical experiment (right). FIG. 7C shows bone matrix quantification (upper panels) three days after L1 RNA (OS+L1) or random RNA sequence (OS) delivery in 4 OP patients derived MSC (HUK7, HUK9, HUK12, HUK16). N=12 technical replicates for each condition for each patient. RFU=Relative Fluorescence Units

FIGS. 8A-F, left panels: Flow cytometer analysis of MSC 6 hours after the delivery of increasing doses of Cy5-L1 RNA in a 6-well plate. Right panels: images of cells 48 hours after Cy5-L1 RNA delivery. The highest dose with minimal toxicity (red rectangle) was selected for the experiments.

FIG. 8G shows the level of apoptotic gene BAX (BCL-2 associated X) and the Interferon-mediated response genes IFNa2 (Interferon Alpha 2), IFNb1 (Interferon Beta 1), IF144 (Interferon Induced Protein 44) in undifferentiated (MSC+L1) and differentiated (OS+L1) cells compared to non-transfected cells (MSC) 72 h post transfection.

FIG. 9A shows Timeline of intracellular lipid accumulation quantified by relative fluorescence (RFU, 485/572). FIG. 9B shows PPARγ (Peroxisome proliferator-activated receptor gamma); FABP4 (Fatty acid binding protein 4); LPL (Lipoprotein lipase); FASN (Fatty acid synthase).

FIG. 10 shows serum TRAP5B correlated to total body bone mineral density (BMD) in the extended cohort of 99 postmenopausal women divided into three groups: healthy (CTR), osteoporotic (OP) and with intermediate phenotype (INTERMEDIATE).

FIG. 11A shows the expression of the three-active murine L1 subfamilies (L1-Tf, L1-Gf and L1-Af) measured in tail tip fibroblasts (TTFs) isolated from wild-type (WT, left bar in each bar pair) and LAKI mice. FIG. 11B shows fluorescence intensity for L1 expression confirmed using an RNA Fluorescent in situ hybridization assay (FISH). FIGS. 11C-11D show L1 RNA depletion confirmed by qPCR and RNA FISH; L1-AON (right bar in each pair of bars). FIG. 11E shows the effect of LAKI TTFs treated with L1-AON on the expression of stress response genes in p53 tumor suppressor pathway (p16, p21, Atf3 and Gadd45b), senescent-associated metalloprotease Mmp13 and proinflammatory interleukin IL1a. FIG. 11F shows the number of cells positive for active senescence-associated β-galactosidase enzyme (SA-B-gal) is reduced in LAKI TTFs treated with L1-AON.

FIGS. 12A-12B shows levels of H3K9me3 in wt, compared to scrbl L1 AON (left bar in each pair of bars, for 12B) and L1-AON (right bar in each pair of bars for 12B) treatment and the effect of L1-AON treatment, on the intensity of H3K9me3 heterochromatin foci in LAKI cells compared to scramble treated control cells and wt (FIG. 12A), and the number of cells with abnormal nuclei structure (FIG. 12B). RNA Immuno-Precipitation (RIP) was performed and the results showed that both the 5′ end and the 3′ end of the L1 RNA is bound by SUV39H1/2 protein in LAKI TTFs (FIG. 12C). FIG. 12D shows studies to determine if L1 RNA plays an inhibitory role on SUV39H1/2 accumulated in the nucleus of LAKI cells. AnH3K9 specific Histone Methyl Transferase assay was performed using a recombinant SUV39H1/2 protein in the presence of the L1 sense-oriented transcript. L1 antisense transcript was used as a negative control.

FIG. 13A shows the knockdown of L1 RNA in several tissues including skin, tibialis anterior skeletal muscle, liver, kidney, spleen and stomach as confirmed by qPCR. FIG. 13B shows the effect of L1-AON treatment (right bar of each bar pair) on the expression of SASP genes in different tissues analyzed. FIG. 13C shows the effect L1-AON (right bar of each bar pair) on the histological profile of skin, spleen, and kidney in mice. FIGS. 13D and 13E show the effect of L1-AON (right bar of each bar pair) treatment on bodyweight (FIG. 13D) and the lifespan (FIG. 13E) of treated mice.

FIG. 14A is a qPCR showing the expression of LINE-1 Ta elements in human Wt (left bar), Progeria syndrome (HGPS, middle bar) and WRN−/− cells (right bar).) N=3. S.E.M and T-Test are showed in the plot. FIG. 14B shows the result of an Sa-B-Gal assay showing the number of senescent cells in HGPS and WRN−/− cells treated with L1-AON (right bar for each pair of bars) and control (left bar for each pair of bars). The plot shows the quantification of the assay. N=6. S.E.M. is showed in the plot. FIG. 14C results from a qPCR showing the expression of the senescence associated genes in HGPS cells after L1-AON treatment (bar to the right of each bar pair). N=6. S.E.M and T-Test are showed in the plot. FIG. 14D results from a qPCR showing the expression of the senescence associated genes in WRN −/− cells after L1-AON treatment (bar to the right of each bar pair). N=6. S.E.M and T-Test are showed in the plot. FIG. 14E is a plot showing H3K9me3 intensity for HGPS cells treated with L1-AON and LAKI control cells. Single replicates, S.E.M and T-Test are showed in the plot. FIG. 14F is a plot showing H3K9me3 intensity WRN−/− cells treated with L1-AON and LAKI control cells. Single replicates, S.E.M and T-Test are showed in the plot.

### DETAILED DESCRIPTION OF THE INVENTION

The disclosed compositions and methods are based on the discovery that L1 mobilization is supported by other tissues, and if L1 expansion may contribute to tissue homeostasis is largely unexplored. A typical L1 element is approx. 6,000 base pairs long and consists of two non-overlapping open reading frames (ORF) which are flanked by untranslated regions (UTR) and target site duplications. L1 has a 5′ untranslated region (UTR) followed by an open reading frame 1 (ORF1), an inter-ORF region, an open reading frame 2 (ORF2) and a 3′ UTR with a poly A site and an associated polyA tail. In humans, ORF2 is thought to be translated by an unconventional termination/reinitiation mechanism. The 5′ Untranslated region (UTR) of the L1 element contains a strong, internal RNA Polymerase II transcription promoter in sense. L1 transcription generates full-length mRNAs that produce two proteins, ORF1p and ORF2p. The first ORF encode a 500 amino acid −40 kDa protein that lacks homology with any protein of known function. The second ORF of L1 encodes a protein that has endonuclease and reverse transcriptase activity.

The disclosed compositions and methods modulate cellular levels of L1, in specific embodiments belonging to the L1HS (L1 human specific) Ta (Transcribed, subset a) subfamily. The Ta (transcribed, subset a) subfamily of L1 LINEs (long interspersed elements) is characterized by a 3-bp ACA sequence in the 3′ untranslated region and contains ˜520 members in the human genome.

### I. Definitions

“Cosmetic composition” as used herein, refers to a composition for topical application to skin or hair of mammals, especially humans. Such a composition may be generally classified as leave-on or rinse off, and includes any product applied to a human body for improving appearance or general aesthetics.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating the symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “host cell” refers to a cell into which a recombinant vector can be introduced.

As used herein, “transformed” and “transfected” encompass the introduction of a nucleic acid (e.g. a vector) into a cell by a number of techniques known in the art.

“Effective amount” and “therapeutically effective amount,” used interchangeably, as applied to the nanoparticles, therapeutic agents, and pharmaceutical compositions described herein, mean the quantity necessary to render the desired therapeutic result. For example, an effective amount is a level effective to treat, cure, or alleviate the symptoms of a disease for which the composition and/or therapeutic agent, or pharmaceutical composition, is/are being administered.

The terms “inhibit” and “reduce” means to reduce or decrease in activity or expression. This can be a complete inhibition or reduction of activity or expression, or a partial inhibition or reduction. Inhibition or reduction can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

### II. Compositions and Methods of Increasing L1 RNA in a Subject

The disclosed compositions and methods are based on the discovery that reduced L1 RNA expression, leading to reduced transposition, in human bone marrow mesenchymal stem cells (hBMSCs) differentiating into bone cells strongly impairs the capability of the cells to produce mineralized bone matrix. Increasing the amount of L1 RNA (preferably in humans, L1HS-Ta1) in bone marrow mesenchymal cells of subjects such as postmenopausal women will counteract the bone loss, thus reducing symptoms associated with osteoporosis. L1-driven structural variations correlate strongly with bone mass and specifically distinguish bone genome and bone mass of healthy vs osteoporotic postmenopausal women. In vitro cell culture experiments disclose the mechanism: 1) L1 genomic expansion contributes positively to bone formation in developing osteoblasts developed from MSC, 2) hampered L1 retrotransposition in developing osteoblasts leads to lack of activation of osteogenic program and reduced mineralization, 3) the mobilization of L1 is specific for osteogenesis, since it does not happen during differentiation of adipocytes from MSC, the accumulating cell type in osteoporotic patients. The present studies also show that even moderate depletion of L1 RNA in differentiating osteoblast is sufficient to induce a significant reduction in the expression of the osteoblasts-related transcription factors.

Further, ORF2, the enzyme encoded by active L1 and necessary for L1 transposition, is an off-target of Nucleoside reverse transcriptase inhibitors (NTRI) used in antiretroviral therapies. Marked reduction in bone mineral density leading to osteoporosis is a major complication in patients treated with NRTI. Compositions and methods disclosed in some embodiments are based on the discovery that NRTI treatment of HBMSCs differentiating into bone cells prevents L1 retrotransposition leading to reduced bone mineralization.

Thus one embodiment discloses methods for increasing L1 RNA in bone progenitor cells, in a subject in need thereof. Exemplary subjects include patients with osteoporosis or with conditions in need of bone regrowth/increasing bone mass index. A second embodiment discloses compositions for increasing L1 RNA in bone progenitor cells in a subject in need thereof. The compositions include nucleic acids enclosing L1 RNA, L1 RNA and optionally, small molecules known to upregulate L1 retrotransposition.

### A. Osteoporosis and Conditions in Need of Increasing Bone Mass Index

Primary osteoporosis is a skeletal disease predisposing to low impact fractures by reducing bone density and destroying its microarchitecture. The skeleton has a strong genetic predisposition since 70-80% of BMD is heritable (1)(2). Primary osteoporosis has a multifactorial origin to which both genes and environment contribute (3). In osteoporosis, the MSC pool of the bone marrow niche promotes the development of adipocytes at the expense of bone building osteoblasts. This mechanism, alone or together with increased bone resorption rate, results in net bone loss (4)(5). Osteoporosis is a major cause of morbidity, mortality and decreased quality of life worldwide (6), leading to more than 8.9 million fractures annually (7). A marked reduction in BMD, increased skeletal fragility and risk of fracture is also a pivotal clinical problem for human immunodeficiency virus (HIV) infected individuals of all ages under NRTI-based ART.

Bone is constantly changing—that is, old bone is removed and replaced by new bone. During childhood, more bone is produced than removed, so the skeleton grows in both size and strength. For most people, bone mass peaks during the third decade of life. By this age, men typically have accumulated more bone mass than women. After this point, the amount of bone in the skeleton typically begins to decline slowly as removal of old bone exceeds formation of new bone.

Men in their fifties do not experience the rapid loss of bone mass that women do in the years following menopause. By age 65 or 70, however, men and women lose bone mass at the same rate, and the absorption of calcium, an essential nutrient for bone health throughout life, decreases in both sexes. Excessive bone loss causes bone to become fragile and more likely to fracture. There are two main types of osteoporosis: primary and secondary. In cases of primary osteoporosis, either the condition is caused by age-related bone loss (sometimes called senile osteoporosis) or the cause is unknown (idiopathic osteoporosis). The term idiopathic osteoporosis is typically used only for men younger than 70 years old; in older men, age-related bone loss is assumed to be the cause. The majority of men with osteoporosis have at least one (sometimes more than one) secondary cause. In cases of secondary osteoporosis, the loss of bone mass is caused by certain lifestyle behaviors, diseases, or medications. Some of the most common causes of secondary osteoporosis in men include exposure to glucocorticoid medications, hypogonadism (low levels of testosterone), alcohol abuse, smoking, gastrointestinal disease, hypercalciuria, and immobilization.

Other conditions in which intervention methods for increasing bone mass index can be useful include spinal fusion therapy, in which autograft or a bone graft, alone or in combination with cells, is delivered to a spinal fusion site (typically, a site between two vertebrae) to treat conditions such as degenerative disk disease, spondylolisthesis, spinal stenosis, scoliosis, Fractured vertebra, Infection, herniated disk and tumor. The intervention is aimed at encouraging bone growth and eventual fusion of the vertebrae between which the spinal fusion therapy is inserted. The compositions disclosed in the present application can be combined with standard spinal fusion therapy to improve bone growth at the site. The disclosed compositions can also be used as adjunctive therapy for fracture healing, especially in the elderly.

### B. Methods for Increasing L1 RNA in a Subject in Need Thereof

The disclosed methods in one embodiment include the providing to a subject in need thereof, bone progenitor cells such as osteogenic bone marrow-derived cells genetically engineered ex vivo to upregulate L1 RNA or gene therapy to increase cellular amount of L1. The methods include in other embodiments, providing L1 RNA or genes encoding L1 RNA to a subject in need thereof, alone or in combination with providing genetically engineered osteogenic bone marrow derived cells as disclosed herein.

L1 RNA can be synthetized in vitro and then introduced into cells of interest, in vitro or in vivo, or, the host cells can be engineered to induce the expression of L1 RNA from L1 genes under certain conditions. One approach includes nucleic acid transfer into primary cells in culture followed by transplantation (preferably, autologous) of the ex vivo transformed cells into the host, either systemically or into a particular organ or tissue. Exemplary subjects include post-menopausal women, subjects diagnosed with osteoporosis, subjects on antiretroviral therapy, for example, NRT1.

In one embodiment, the disclosed compositions contain the sequence of the human L1 RNA (L1-Ta subfamily), alone, or in a vector, transferred into primary cells.

(SEQ ID NO: 1)
CCATAAGAAACTTTTTAAAAATAAAAAAAACTATAATAAA
AATTATAAGACACTGTAAATGAATGGTTTTAGTCATCTGAATTTT
CTCTGACCCTTAGAATTAAGGAAAAAAAAAACACACAATAGTTAT
GGCCAATAATAATTCCTCCTTCTTTGAGGAAATTCAACAGTTTTA
GAACCAACTTTAAACTATACAACCTGGCATATACACATTTTTAAA
AACACACACACAAAATTGTTTACTAAGCAAGAAGGAGAATTCATT
AAAGAGCCACGCCAGCTACAGGAAGTTTAGATTTTAAATGTAAAG
AGAGGAGGCAATGGTACCTGACTTTTCACATTTTGATTTATATTG
GCTTCTTTCCAGAAAACGCTTTCACTGGGTTTCAAAAAATTTTTA
TTTTATCTCCAACCCTTAGACGGAAACCATCTTCAACTGGAAATC
AGAACACCTAGGTTTTAGTTTTTATTTAGGCAAAAATGTATTTGG
AGCCAGGCATGGTGGATCACAACTGTAATCCCAGCAGTTTGGGAG
GCCAAGGTGGGCAGATAATGTGTGGTCAGGAGTTCGAGATGAGCC
TGGCCAACATGGCAAAACCCCATCTCTAGTAAAAATATAAAAATT
AGCCAGGTGTGGTGTTGCATGCGTGTGGTCTCAGGTACTTGGAAG
GCTGAGGCAGGAGAATCACTTAAGCCCAGGAAACGGAGGTTGCAG
TGAGCCAAGATCACACCATTGCACTGGGCGACAGAGCGAGACTCC
ATCAAAAATAATAATAATGAAATAAATAAATAAATAAAAAGCACA
TTTGGTCTCCACCTACTGGAAAACAGGCAGGAATTGCTGTGTGTT
TCACAGACATGGACCACGAAGAGGATAAAGTGAGTTAACTTGTTA
AGTGCTAAGAAATATTCTCCATAAACCCAAGCTATTATAGTTACG
TAATATGAGTCTCTTCTCTGGAGGGGGTCTTCATGGATTGTTTGC
CAGCCTAGCCTTAAAAATGTCAGGGATCGAAGATGGCCGAATAGC
AACAGCTCCCGTCTACAGCTCCCAGCGTGAGCGACGCAGAAGACG
GGTGATTTCTGCATTTCCATCTGAGGTACCGGGTTCATGTCACTA
GGGAGTGCGAGACAGTGGGGGCAGGCCAGTGTGTGTGCGGACCGT
GTGCCAGCCGAAGCAGGGCGAGGCATTGCCTCACCTGGGAAGCGC
AAGGGGTCAGGGAGTTCCCTTTCCGAGTGAAAGAAAGGGGTGACC
GAGGCACCTGGAAAATCGGGTGACTCCCACCCGAATATTGCGCTT
TTCAGACCGGCTTAAGAAACGGCGCACCACGAGACTATATCCCAC
ACCTGGCTCAGAGGGTCCTACGCCCACGGAATCTCGCTGATTGCT
AGCACAGCAGTCTGAGATCAAACTGCAAGGCGGCAACGAGGCTGG
GGGAGGGGCGCCCGCCATTGCCCAGGCTTGCTTAGGTAAACAAAG
CAGCCAGGAAGCTCGAACTGGGTGGAGCCCACCACAGCTCAAGGA
GGCCTGCCTGCCTCTGTAGGCTCCACCTCTGGGGGCAGGGCACAG
ACAAACAAAAAGACAGCAGTAACCTCTGCAGACTTAAATGTCCCT
GTCTGACAGCTTTGAAGAGAGCAGTGGTTCTCCCAGCAGGCAGCT
GGAGATCTGAGAACGGGCAGACTGCCTCCTCAAGTGGGTCCCTGA
CCCCTGACCCCCGAGCAGCCTAACTGGGAGGCACCCCCCAGCAGG
GGCACAGTGACACCTCACACGGGAGGGTATTCCAACAGACCTGGA
GCTGAGGGTCCTGTCTGTTAGAAGGAAAACTAACAACCAGAAAGG
ACATCTACACCGAAAACCCATCTGTACATCACCATCATCAAAGAC
CAAAAGTAGATAAAACCACAAAGATGGGGAAAAAACAGAACAGAA
AAACTGGAAACTCTAAAACACAGAGCGCCTCTCCTCCTCCAAAGG
AACGCAGTTCCTCACCAGCAACAGAACAAAGCTGGATGGAGAATG
ATTTTGACGAGCTGAGAGAAGAAGGCTTCAGACGATCAAATTACT
CTGAGCTACGGGAGGACATTCAAACCAAAGGCAAAGAAGTTGAAA
ACTTTGAAAAAAATTTAGAAGAATGTATAACTAGAATAACCAATA
CAGAGAAGTGCTTAAAGGAGCTGATGGAGCTGAAAACCAAGGCTC
GAGAACTACGTGAAGAATGCAGAAGCCTCAGGAGCCGATGCGATC
AACTGGAAGAAAGGGTATCAGCAATGGAAGATGAAATGAATGAAA
TGAAGCGAGAAGGGAAGTTTAGAGAAAAAAGAATAAAAAGAAATG
AGCAAAGCCTCCAAGAAATATGGGACTATGTGAAAAGACCAAATC
TACGTCTGATTGGTGTACCTGAAAGTGATGTGGAGAATGGAACCA
AGTTGGAAAACACTCTGCAGGATATTATCCAGGAGAACTTCCCCA
ATCTAGCAAGGCAGGCCAACGTTCAGATTCAGGAAATACAGAGAA
CGCCAGAAAGATACTCCTCAAGAAGAGCAACTCCAAGAGACATAA
TTGTCAGATTCACCAAAGTTGAAATGAAGGAAAAAATGTTAAGGG
CAGCCAGAGAGAAAGGTCGGGTTACCCTCAAAGGAAAGCCCATCA
GACTAACAGCGGATCTCTCGGGAGAAACCCTACAAGCCAGAAGAG
AGTGGGGGCCAATATTCAACATTCTTAAAGAAAAGAATTTTCAAC
CCAGAATTTGATATCCAGCCAAACTAAGCTTCATAAGTGAAGGAC
AAATAAAATACTTTATAGAGAAGCAAATGCTGAGAGATTTTGTCA
CCACCAGGCCTGCCCTAAAAGAGCTCCTGAAGGAAGCGCTAAACA
TGGAAAGGAACAATCGGTACCAGCCGCTGCAAAATCATGCGAAAA
TGTAAAGACCATCAAGACTAGGAAGAAACTGCATCAACTAATGAG
CAAAATCACCAGCTAACATCATAATGACAGGATCAAATTCACACA
TAAGAATATTAACTTTAAATGTAAATGGACTAAATTCTGCAATTA
AAAGACACAGACTGGCAAGTTGGATAAAGAGTCAAGACCCATCAG
TGTGGTGTATTCAGGAAACCCATCTCGCGTGCAGAGACACACATA
GGGTCAAAATAAAAGGATGGAGGAAGATCTACCAAGGGAATGGAA
AACAAAAAAAGGCAGGGGTTGCAATCCTAGTCTCTGATAAAACAG
ACTTTAAACCAACAAAGATCAAAAGAGACAAAGAAGGCCATTACA
TAATGGTAAAGGGATGAATTCAACGAGAGGAGCTAACTATCCTAA
ATATTTATGCACCCAATACAGGAGCACCCAGATTCATAAAGCAAG
TCCTGAGTGACCTACAAAGAGAGTTAGACTCCCACAGATTAATAA
TGGGAGACTTTAACACCCCACTGTCAACATTAGACAGATCAACGA
GACAGAAAGTCAACAAGGATACCCAGGAATTGAACTCAGCTCTGC
ACCAAGCAGACCTAATAGACATGTACAGAACTCTCGACCCCAAAT
CAACAGAATATACATTTTTTTCAGCACCACACCACACCTATTCCA
AAATTGACCACATAGTTGGAAGTAAAGCTCTCCTCAGCAAATGTA
AAAGAACAGAAATTATAACAAACTATCTCTGAGACCACAGTGCAA
TCAAACTAGAACTCAGGATTAAGAATCTCACTCAAAGCCGCTCAA
CTACATGGAAACTGAACAACCTGCTCCTGAATGACTACTGGGTAC
ATAACGAAATGAAGGCAGAAATAAAGATGTTCTTTGAAACCAAGG
AGAACAAAGACACCACATACCAGAATCTCTGGGACGCATTCAAAG
CAGTGTGTAGAGGGAAATTTATAGCACTAAATGCCCACAAGAGAA
AGCAGGAAAGATCCAAAATTGACACCCTAACATGACAATTAAAAG
AACTAGAAAAGCAAGAGCAAACACATTCAAAAGCTAGCAGAAGGC
AAGAAATAACTAAAATCAGAGCAGAACTGAAGGAAATAGAGACAC
AAAAAACCCTTCAAAAAATCAATGAATCCAGGAGCTGGTTTTTTG
AAAGGATCAACAAAATTGATAGACCGCTAGCAAGACTAATAAAGA
AAAAAAGAGAGAAGAATCAAATAGACACAATAAAAAATGATAAAG
GGGATATCACCACCGATCCCACAGAAATACAAACTACCATCAGAG
AATACTACAAACACCTCTACGCAAATAAACTAGAAAATCTAGAAG
AGATGGATACATTCCTCGACACATACACTCTCCCAAGACTAAACC
AGGAAGAAGTTGAATCTCTGAATAGACCAATAACAGGCTCTGAAA
TTGTGGCAATAATCAATAGTTTACCAACCAAAAAGAGTCCAGGAC
CAGATGGATTCACAGCCGAATTCTACCAGAGGTACATGGAGGAAC
TGGTACCATTCCTTCTGAAACTATTCCAATCAATAGAAAAAGAGG
GAATCCTCCCTAACTGATTTTATGAGGCCAGCATCATTCTGATAC
CAAAGCCGGGCAGAGACACAACCAAAAAAGAGAATTTTAGAACAA
TATCCTTGATGAACATTGATGCAAAAATCCTCAATAAAATACTGG
CAAACCGAATCCAGCAGCACATCAAAAAGCTTATCCACCATGATC
AAGTGGGCTTCATCCCTGGGATGCAAGGCTGGTTCAATATACGCA
AATCAATAAATGTAATCCAGCATATAAACAGAGCCAAAGACAAAA
ACCAGATGATTATCTCAATAGATGGAGAAAAAGCCTTTGACAAAA
TTCAACAACCCTTCATGCTAAAAACTCTCAATAAATTAGGTATTG
ATGGGATGTATTTCAAAATAATAAGAGCTATCTATGACAAAGCCA
CAGGGAATATCATTCTGAATGGGCAAAAACCGGAAACATTCCCTT
TGAAAATTGGCACAAGACAGGGATGCCCTCTCTCACCGCTCCTAT
TCAACATAGTGTTGGAAGTTCTGGGGAGGGCAATCAGGCAGGAGA
AGGAAATAAAGGGTATTCAATTAGAAAAAGAGGAAGTCAAATTGT
CCCTGTTTGCAGACGACATGATTGTTTATCTAGAAAACCCCATCG
TCTGAGCCCAAAATCTCCTTAAGGTGATAAGCAACTTCAGCAAAG
TCTCAGGATACAAAATCAATGTACAAAAATCACAAGCATTCTTAT
ACACCAACAACAGAGAAACAGAGAGCCAAATCATGAGTGAACTCC
CATTCACAATTGCTTCAAAGAGAATAAAATACCTAGGAATCCAAC
TTACAAGGGATGTGAAGGACCTCTTCAAGGAGAACTACAAACCAC
TGCTCAAGGAAATAAAAGAGGACACAAACAAATGGAAGAACATTC
CATGCTGATGGGTAGGAAGAATCAATATCGTGAAAATGGCCATAC
TGCCCAAGGTAATTTACAGATTCAATGCCATCCCCATCAAGCTAC
CAATGACTTTCTTCACAGAATTGGAAAAAACTACTTTAAAGTTCA
TATGGAACCAAAAAAGAGCCCGCATCGCCAAGTCAATCCTAAGCC
AAAAGAACAAAGCTGGAGGCATCACACTACCTGACTTCAAACTAT
ACTAGAAGGGTACAGTAACCAAAACAGCATGGTACTGGTACCAAA
ACAGAGATATAGAGCAATGGAACAGAAGAGAGCCCTCAGAAATAA
TGCCACATATCTACAACTATCTGAGCTTTGACAAACCTGAGAAAA
ACAAGCAATGGGGAAAGGATTCCCTATTTAATAAATGGTGCTGGG
AAAACTGGCTGGCCATATGTAGAAAGCTGAAACTGGATCCCTTCC
TTACACCTTATACAAAAATTAATTCAAGATGGATTAAAGATTTAA
ACGTTAGACCTAAAACCATAAAAACCCTAGAAGAAAACCTAGGCA
TTACCATTCAGGACATAGGCGTGGGCAAGGACTTCATGTCTAAAA
CACCAAAAGCAATGGCAACAAAAGCGAAAATTGACAAATGGGATG
TAATTAAACTAAAGAGCTCCTGCACAGCAAAAGAAACTACCATCA
GAGAGAACAGGCAACCTACAACATGGGAGAAAATTTTCGCAACCT
ACTCATCTGAGAAAGGGCTAATATGGAGAATCTACAATGAACTGA
AACAAATTTACAAGAAAAAAACAAACAACCCCATCAAAAAGTGGG
CGAAGGACATGAAGAGACACTTCTCAAAAGAAGACATTTATGCAG
CCAAAAAACACATGAAAAAATGCTGACCATGACTGGCCATCAGAG
AAATGCAAATCAAAACCACAATGAGATACCATCTCACACCAGTTA
GAATGGCAATCATTAAAAAGTCAGGAAACAAGAGGTGGTGGAGAG
GATGTGGAGAAATAGGAACACTTTTACACTGTTGGTGGGACTGTA
AACTAGTTCAACCATTGTGGAAGTCAGTGTGGGGATTCCTCAGGG
ATCTAGAACTAGAAATACCATTTGACCCAGCGATCCCATTACTGG
GTATATACCCAAAGGACTATAAATCATGCTGCTATAAAGAGACAT
GCACATGTATGTTTATTGCGGCACTATTCACAATAGCAAGGACTT
GGAACCAACCCAAATGTCCAACAATGATAGACTGGATTAAGAAAA
TGTGGCACATATACACCATGGAATACTATGCAGCCATAAAAAATG
ATGAGTTCATGTCCTTTGTAGGGACATGGATGAAATTGGAAACCA
TCATTCTCAGTAAACTATCACAAGAACAAAAAACCAAACACCGCA
TATTCTCACTCATAGGTGGGAATTGAACAATGAGATCACTTGGAC
ACAGGAAGGGGAATATCACACTCTGGGGACTGTTGTGGGGTGGGG
GGAGGGGGGAGGGATAGGATGGGAGATATACCTAATGCTAGATGA
CACGTTAGTGGGTGCAGCGCACCAGCATGGCACATGTATACATAT
GTAACTAACCTGCACAATGTGGACATGTACCCTAAAACTTAGAGT
ATAATAAAAAAAAAAAAATTTAAAAAAAAAAAAAAAATGCAAGTT
TAGTTAACATTTTCAGCCAGCATGCTAAACCTTGTGATGAAGTCA
TAGGGTCTTATACTCACGATTTGATAACATAATAGAACTATTAAA
AGAAGTCTGATTCATTAAAAAAAAAAAAAAATGTCAGGCATCACA
AACAGGGAAACCTATAAATGAGAAATTTGGCTGCAGTCAGTCCCA
GGGGACTCTCTGGCACTGGTGCTGTGGGGACTCACAGTTTAATCA
GTTAAATCTGCATCAGGAAGTCAGCTCCCCTTCTGCTGCACCCGC
TGGTGTCACAGAGGTCCTGAGCATCAAGCCAACACCTCCTGCTGA
GTAGCAAAACGCTTGTGCCACCTGCCATCCTGGGGAAAACAGCCA
TGCTGCTGGAGAGTGTAATCAATGGAGAGAGAGCCCGGTCACTTC
TGCCATCTTGCTCTCCTCCCACCTGGGAATAGAAGTGACAACTAT
TGAGCCATCAGAATGAAAATCTTTACTAGCCGTGCAGTGATCGAG
GTACATAGAGTGACCTGCAACACTACAGTTTTAGGAAGGATATTG
ATTGTCTTATCTAACAGGAAAATGTGAAGGGAGTTTATATTTTAG
GTTAACTCTGTCTCAGGCTTTCTCACTTTGGTTCTTTCACACATA
TTCAACAAACATCCATTGTCTGTAATGTTCCAGGCACTGTTCTAG
GTCATATGAAAAATCAGAATTGAACAGACATAGCCACTTACTCTC
AAAGAGTTTACTATTCAGTAAAAGGAAAGAAGATAAGTGCAATAT
AAAGTTATAATGATACAAGACAGGTGTGTCTATTTGCATATATAT
TTATATAAAAATGAATAAAATCTTGAGGCTATTTTTATTTAATAA
CGTCTCAGGATACTATAAGAGGTGAGGAGAAGGGCCAATGGATTG
AGATTCACTAATGACTCAAGGTATGATAATTACTATTCTCTGTTG
TTTTTAAAAAAACTTTTCTTCATGTAATGATOTACGGGCATTTTT.

However, the composition can include fragments of L1 RNA, for example. The L1 open reading frame 1 (ORF1), alone or preceded by a 5′ untranslated region (UTR), or the an open reading frame 2 (ORF2), ORF2 expression constructs are disclosed for example, in Gasior, et al., J. Mol. Biol., 357 (5): 1383-1393 (2006).

### I. Ex Vivo Methods

Ex vivo methods can include, for example, the steps of harvesting cells from a subject, culturing the cells, transducing them with an expression vector including DNA encoding L1 RNA or L1 RNA, and maintaining the cells under conditions suitable for expression of the encoded RNA. These methods are known in the art of molecular biology. In a preferred embodiment, the cells are autologous to the subject being treated. A preferred host cells are hBMSCs. Methods for isolating hBMSC are known in the art (Baghaevi, et al., Gastroenterol Hepatol Bed Bench, 10 (3): 208-2013 (2017).

### I. Vectors

Vectors encoding L1 RNA also provided. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Hamann, et al., J. Biol. Eng., 13:7 (2019) demonstrated that gene expression in hBMSCs driven by cytomegalovirus (CMV) promoter, resulted in 10-fold higher transgene expression than transfection with plasmids containing elongation factor 1 α (EF1α) or rous sarcoma virus (RSV) promoters.

Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA). Recent transfection studies have investigated minicircle DNA (mcDNA), nucleic acids that are derived from pDNA by recombination that removes bacterial sequences. L1 RNA can be introduced into host cells using mcDNA using methods known in the art (Mun et al. Biomaterials, 2016; 101:310-320).

### 2. Host Cell Transformation

Vectors containing nucleic acids to be expressed can be transferred into host cells. The term “host cell” is intended to include bone progenitor cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Nucleic acids can be transfected into mammalian cells by techniques including, for example, calcium phosphate co-precipitation, DEAE-dextran-mediated transfection, lipofection, electroporation, or microinjection. Preferred host cells include bone progenitor cells such as HBMSC or osteoblasts.

The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject.

Effective strategies for nonviral transfection of MSCs ex vivo typically employ disruption of cell membranes to transfer nucleic acids into cells (e.g. microinjection, electroporation, and microporation) or packaging of nucleic acids with nanocarrier materials that facilitate cellular internalization through endocytosis.

The primary alternative to electroporation for nucleic acid transfer into MSCs ex vivo is transfection with nanocarriers, materials that electrostatically condense or encapsulate nucleic acids into nanoparticles or aggregate complexes that favorably associate with cell membranes through charge interactions or surface receptor binding, and are subsequently internalized via macropinocytosis, clathrin-mediated endocytosis, or caveolae-mediated endocytosis, depending primarily on nanoparticle size and charge. Carriers have been demonstrated to facilitate transfection of MSCs, including, but are not limited to, polymers, lipids, polysaccharides, peptides, and inorganic materials. Examples include, but are not limited to nano-hydroxyapatite (nHA), the ubiquitous cationic polymer transfection reagent 25 kDa branched polyethylenimine (bPEI), preferably functionalized with hyaluronic acid, and repeating arginine-alanine-leucine-alanine (RALA) amphipathic peptide, poly(amidoamine)(PAMAM), poly(β-amino-esters)(PBAE), PEI-coated PLGA nanoparticles etc., reviewed in Hamann, et al., J. Biol. Eng., 13:7 (2019).

Cell culture conditions for improving transcription efficiency can be used to ensure efficient uptake of the nucleic acid being introduced into the cell. For example, glucocorticoids (Gc) can dramatically enhance transfection in MSCs ex vivo. 100 nM of the Gc dexamethasone (DEX) delivered 0-30 min prior to transfection was shown to increase transgene expression in hBMSCs.

Transformed bone progenitor cells are preferably separated and cultured in GMP conditions to purify and obtain an established dose range.

ii. In Vivo Methods

In vivo methods include introducing engineered bone progenitor cells as disclosed herein into a subject in need thereof, or direct transfer of L1 RNA or DNA encoding L1 RNA into a subject in need thereof. The disclosed methods can also include administering to the subject small molecules and compounds known to upregulate L1 RNA transcription and retrotransposition. For example, agents such as benzo[a]pyrene, camptothecin, cytochalasin D, merbarone, and vinblastine; PPARα agonists (bezafibrate and fenofibrate), and non-steroidal anti-inflammatory drugs (diflunisal, flufenamic acid, salicylamide, and sulindac) have been shown to induce L1 promoter activity (Terasaki, et al., PLoS One. 2013; 8 (9): e74629.

The cells (genetically engineered to include a vector containing L1 RNA or DNA encoding L1 RNA) can be introduced into the subjects using method known in the art, for example, intravenously. Autologous transformed BMSC can be infused intravenously at dosing ranging from a dose of 2 million cells/Kg to 5 million cells/kg. In embodiments where the cells are delivered intravenously, on the infusion day the transformed cells are re-suspended in saline to a concentration of 5 million cells per 1 mL and preferably, fucosylated. Then, the final product can be packaged in syringes for intravenous administration to patients through a peripheral venous access. Methods for improving homing of hBMSC to the bone marrow using fucosyltransferases are known in the art. Essentially, exogenously introduced fucosyltransferases are used to modify CD44 expressed by MSCs into HCELL (hematopoietic cell E-/L-selectin ligand), a potent E-selectin ligand critical for HSC homing to the bone marrow. Essentially, exogenously introduced fucosyltransferases are used to modify CD44 expressed by MSCs into HCELL (hematopoietic cell E-/L-selectin ligand), a potent E-selectin ligand critical for HSC homing to the bone marrow (reviewed in Krueger, et al., Stem Cells Translational Med., 7:651-663 (2018).

In vivo gene therapy can be employed, whereby the genetic material is transferred directly into the patient. In these embodiments, genetic material is introduced into a patient by a virally derived vector or by non-viral techniques. In vivo nucleic acid therapy can be accomplished by direct transfer of a functionally active DNA into mammalian somatic tissue or organ in vivo. Nucleic acids be administered in vivo by viral means. A therapeutic gene expression cassette is typically composed of a promoter that drives gene transcription, the transgene of interest, and a termination signal to end gene transcription. Such an expression cassette can be embedded in a plasmid (circularized, double-stranded DNA molecule) as delivery vehicle. Plasmid DNA (pDNA) can be directly injected in vivo by a variety of injection techniques, among which hydrodynamic injection achieves the highest gene transfer efficiency in major organs by quickly injecting a large volume of pDNA solution and temporarily inducing pores in cell membrane. To help negatively charged pDNA molecules penetrate the hydrophobic cell membranes, chemicals including cationic lipids and cationic polymers have been used to condense pDNA into lipoplexes and polyplexes, respectively.

L1 RNA or nucleic acid molecules encoding L1 RNA may be packaged into retrovirus vectors using packaging cell lines that produce replication-defective retroviruses, as is well-known in the art. Other virus vectors may also be used, including recombinant adenoviruses and vaccinia virus, which can be rendered non-replicating. Nucleic acids may also be delivered by other carriers, including liposomes, polymeric micro- and nanoparticles and polycations such as asialoglycoprotein/polylysine. Various techniques and methods for in vivo gene delivery using the disclosed vectors and carriers are known in the art (reviewed in Wang, et al., Discov. Med., 18 (97): 67-77 (2014). A major advancement in DNA vector design is minicircle DNA (mcDNA), which differs from pDNA in the lack of bacteria-derived, CpG-rich backbone sequences. When administered in vivo, mcDNA mediates safer, higher and more sustainable transgene expression than conventional pDNA

### III. Compositions and Methods of Reducing L1 RNA in a Subject

The disclosed methods and applications rely on reducing the level of L1 RNA, nucleic acids encoding line 1 RNA or L1 RNA encoded proteins in a subject in need thereof. The methods and applications are based on the discovery that reduced L1 RNA levels reduce markers of ageing such as markers cell senescence in fibroblasts and health of the skin, for example, thickness of the epidermal layer.

Down regulation of L1 RNA expression can be used to treat conditions associated with ageing for example, progeria syndrome. Hutchinson-Gilford Progeria Syndrome (“Progeria”, or “HGPS”) is a rare, fatal genetic condition characterized by an appearance of accelerated aging in children. Although they are born looking healthy, children with Progeria begin to display many characteristics of accelerated aging within the first two years of life. Progeria signs include growth failure, loss of body fat and hair, aged-looking skin, stiffness of joints, hip dislocation, generalized atherosclerosis, cardiovascular (heart) disease and stroke. Other progeroid syndromes include Werner's syndrome, also known as “adult progeria” which does not have an onset until the late teen years. There is no cure for progeria, but occupational and physical therapy can help the child keep moving if their joints are stiff. The disclosed compositions and methods can ameliorate the accelerated ageing symptoms associated with Progeria Syndrome. The examples below demonstrate that show that depletion of L1 RNA in cells obtained from HGPS mouse model (LAKI) using antisense oligonucleotides (AON) restored the levels of epigenetic marks and reduced the expression of senescent-associated genes, and increased life span.

Down regulation of L1 RNA expression can also find application in cosmetic compositions. In some embodiments the cosmetic compositions can be used topically or subcutaneously to treat the signs of ageing. These signs include formation of fine lines and wrinkles, inadequate skin firmness, reduction of skin luminescence, lack of skin smoothness, poor skin elasticity, formation of age spots, blotching, sallowness, uneven pigmentation and combinations thereof. The compositions are effective in some embodiments to improve thickness of the epidermal layer.

### A. L1 RNA Downregulation/Inhibition

L1 RNA can be downregulated by treating cells to downregulate L1 RNA levels. This step includes contacting the cells with one or more agents to inhibit L1 RNA. Agents that inhibit L1 RNA as used herein include, but at not limited to agents that reduce the retrotransposition of L1 RNA in a cell and agents that inhibit any of the activities of the proteins expressed by L1 RNA. The L1 RNA inhibiting agent can be a nucleic acid, a peptide (for example, a peptide aptamer) or a small molecule.

Compounds that have been found to inhibit Line 1 retrotansposition include, but are not limited to Capsaicin (Nishikawa, et al., Int J Mol Sci. 2018 October; 19 (10): 3243), and three selective line 1 reverse transcriptase inhibitors, GBS-149, emtricitabine and lamivudine, disclosed in Banuelos-Sanchez, et al., Cell Chem. Biol. 26 (8): P1095-1109 (2019).

L1 RNA can be inhibited using a functional nucleic acid (herein, L1 RNA-inhibiting NA), or vector encoding the same, which downregulate expression of L1ORF1, L1-ORF2 or the combination thereof. Examples include, but are not limited to antisense oligonucleotides, siRNA, shRNA, miRNA, EGSs, ribozymes, and aptamers (nucleic acid and peptide aptamers). In a particularly preferred embodiment, L1 RNA is downregulated in a subject in need thereof, using an antisense oligonucleotide, for example, fluoroarabinonucleic acids (FANA) modified antisense oligonucleotides (ASOs) specific for L1-ORF1 RNA sequence L1 RNA-inhibiting ASOs (or vectors expressing the same) can be formulated as described herein and administered to a subject in need thereof.

### I. RNA Interference

In some embodiments, L1 RNA expression is inhibited through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point, the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of RNAi or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). SiRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). SiRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

ii. Antisense

L1 RNA can be inhibited using can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

An “antisense” nucleic acid sequence (antisense oligonucleotide) can include a nucleotide sequence that is complementary to a “sense” nucleic acid encoding a protein, e.g., complementary to the L1 RNA. Antisense nucleic acid sequences and delivery methods are well known in the art (Goodchild, Curr. Opin. Mol. Ther., 6 (2): 120-128 (2004); Clawson, et al., Gene Ther., 11 (17): 1331-1341 (2004). The antisense nucleic acid can be complementary to an entire coding strand of a target sequence, or to only a portion thereof. An antisense oligonucleotide can be, for example, about 7, 10, 15, 20, 25, 30, 35, 40, 45, 50, 55, 60, 65, 70, 75, 80, or more nucleotides in length.

The ASO can be complementary to full length L1RNA, L1 5′UTR, L1RNA ORF1, L1 RNA ORF2 and/or L1 3′UTR. Exemplary antisense oligonucleotides are provided below.

Oligos against L1 5′UTR
(SEQ ID NO: 57)
GTACCTCAGATGGAAATGCAG
(SEQ ID NO: 58)
ATGAACCCGGTACCTCAGATG
(SEQ ID NO: 59)
CCCTAGTGAGATGAACCCGGT
(SEQ ID NO: 60)
GTCTGGCACTCCCTAGTGAGA
(SEQ ID NO: 61)
TGCGCCCACTGTCTGGCACTC
(SEQ ID NO: 62)
CACACTGGCCTGCGCCCACTG
(SEQ ID NO: 63)
GGTGCGCACACACACTGGCCT
(SEQ ID NO: 64)
GCTCGCGCAAGGTGCGCACAC
(SEQ ID NO: 65)
CCCTGCTTCGGCTCGCGCAAG
(SEQ ID NO: 66)
CAATGCCTCGCCCTGCTTCGG
(SEQ ID NO: 67)
CCAGGTGAGGCAATGCCTCGC
(SEQ ID NO: 68)
CTTGCGCTTCCCAGGTGAGGC
(SEQ ID NO: 69)
TCCCTGACCCCTTGCGCTTCC
(SEQ ID NO: 70)
GAAAGGGAACTCCCTGACCCC
(SEQ ID NO: 71)
CTTTGACTCGGAAAGGGAACT
(SEQ ID NO: 72)
TCACCCCTTTCTTTGACTCGG
(SEQ ID NO: 73)
GGTGCGTCCGTCACCCCTTTC
(SEQ ID NO: 74)
CGATTTTCCAGGTGCGTCCGT
(SEQ ID NO: 75)
GGGAGTGACCCGATTTTCCAG
(SEQ ID NO: 76)
ATATTCGGGTGGGAGTGACCC
oligos against L1 ORF1
(SEQ ID NO: 77)
CTTTGTTCTGTTGCTGGTGAG
(SEQ ID NO: 78)
CTCCATCCAGCTTTGTTCTGT
(SEQ ID NO: 79)
CAAAATCATTCTCCATCCAGC
(SEQ ID NO: 80)
CTCAGCTCGTCAAAATCATTC
(SEQ ID NO: 81)
GCCTTCTTCTCTCAGCTCGTC
(SEQ ID NO: 82)
ATCGTCTGAAGCCTTCTTCTC
(SEQ ID NO: 83)
GAGTAATTTGATCGTCTGAAG
(SEQ ID NO: 84)
CCGTAGCTCAGAGTAATTTGA
(SEQ ID NO: 85)
GAATGTCCTCCCGTAGCTCAG
(SEQ ID NO: 86)
CCTTTGGTTTGAATGTCCTCC
(SEQ ID NO: 87)
AACTTCTTTGCCTTTGGTTTG
(SEQ ID NO: 88)
CAAAGTTTTGAACTTCTTTGC
(SEQ ID NO: 89)
AAATTTTTTTCAAAGTTTTGA
(SEQ ID NO: 90)
ACATTCTTCTAAATTTTTTTC
(SEQ ID NO: 91)
TTCTAGTTATACATTCTTCTA
(SEQ ID NO: 92)
GTATTGGTTATTCTAGTTATA
(SEQ ID NO: 93)
GCACTTCTCTGTATTGGTTAT
(SEQ ID NO: 94)
GCTCCTTTAAGCACTTCTCTG
(SEQ ID NO: 95)
AGCTCCATCAGCTCCTTTAAG
(SEQ ID NO: 96)
CTTGGTTTTCAGCTCCATCAG
oligos against L1 ORF2
(SEQ ID NO: 97)
TGTTATGTGTGAATTTGATCC
(SEQ ID NO: 98)
AAGTTAATATTGTTATGTGTG
(SEQ ID NO: 99)
TTTATATTTAAAGTTAATATT
(SEQ ID NO: 100)
ATTTAGTCCATTTATATTTAA
(SEQ ID NO: 101)
TAATTGCAGAATTTAGTCCAT
(SEQ ID NO: 102)
CTGTGTCTTTTAATTGCAGAA
(SEQ ID NO: 103)
ACTTGCCAGTCTGTGTCTTTT
(SEQ ID NO: 104)
TCTTTATGCAACTTGCCAGTC
(SEQ ID NO: 105)
GGGTCTTGACTCTTTATGCAA
(SEQ ID NO: 106)
GCACACTGATGGGTCTTGACT
(SEQ ID NO: 107)
TGTGGGATCGGTGGTGATATC
(SEQ ID NO: 108)
TTTGTATTTCTGTGGGATCGG
(SEQ ID NO: 109)
CTGATGGTAGTTTGTATTTCT
(SEQ ID NO: 110)
GTAGTATTCTCTGATGGTAGT
(SEQ ID NO: 111)
AGAGGTGTTTGTAGTATTCTC
(SEQ ID NO: 112)
TTATTTGCGTAGAGGTGTTTG
(SEQ ID NO: 113)
ATTTTCTACTTTATTTGCGTA
(SEQ ID NO: 114)
TTTCTTCTAGATTTTCTACTT
(SEQ ID NO: 115)
AATGTATCCATTTCTTCTAGA
(SEQ ID NO: 116)
TGTGTCGAGGAATGTATCCAT
oligos against L1 3′UTR
(SEQ ID NO: 117)
TAGCATTAGGTATATCTCCCA
(SEQ ID NO: 118)
ATGTGTCATCTAGCATTAGGT
(SEQ ID NO: 119)
GCACCCACTAATGTGTCATCT
(SEQ ID NO: 120)
CTGGTGCGCTGCACCCACTAA
(SEQ ID NO: 121)
ATGTGCCATGCTGGTGCGCTG
(SEQ ID NO: 122)
ATATGTATACATGTGCCATGC
(SEQ ID NO: 123)
GGTTAGTTACATATGTATACA
(SEQ ID NO: 124)
ACATTGTGCAGGTTAGTTACA
(SEQ ID NO: 125)
GTACATGTGCACATTGTGCAG
(SEQ ID NO: 126)
AAGTTTTAGGGTACATGTGCA
(SEQ ID NO: 127)
ATTATACTCTAAGTTTTAGGG

The OSA can be a locked-nucleic-acid (LNA)-modified ASO. LNA ASOs have been used in many different settings such as antisense gapmers, anti-microRNAs (antagomiRs), and anti-gene approaches. An LNA is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA designs can be divided in two main categories: mixmers and gapmers. In a mixmer, LNA and DNA nucleosides are interspersed throughout the sequence of the oligonucleotide, whereas, in a gapmer, two LNA segments at both ends of the oligonucleotide are separated by a central segment or gap of DNA nucleosides. Gapmers are preferred for RNA inhibition. This is because the central DNA/PS segment, which is longer than 7-8 DNA nucleotides (nt), recruits the RNA-cleaving enzyme RNase H when the gapmer is hybridized to the mRNA.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Other examples of useful antisense oligonucleotides (AONs/ASOs) include an alpha-anomeric nucleic acid. An alpha-anomeric nucleic acid molecule forms specific double-stranded hybrids with complementary RNA in which, contrary to the usual beta-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

A particularly preferred antisense oligonucleotide (ASO) is the fluoroarabinonucleic acids (FANA) modified ASOs specific for L1-ORF1 RNA sequence. FANA ASOs bind the target sequence and act as docking elements for RNAseH-mediated cleavage.

### 1. Aptamers

In some embodiments, the inhibitory molecule is an aptamer. Aptamers are molecules that interact with a target molecule, preferably in a specific way. Aptamers can bind the target molecule with a very high degree of specificity. For example, aptamers have been isolated that have greater than a 10,000 fold difference in binding affinities between the target molecule and another molecule that differ at only a single position on the molecule. Because of their tight binding properties, and because the surface features of aptamer targets frequently correspond to functionally relevant parts of the protein target, aptamers can be potent biological antagonists. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

### 2. Ribozymes

L1 RNA expression can be inhibited using ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

### 3. Triplex Forming Oligonucleotides

L1 RNA expression can be inhibited using triplex forming molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

### 4. External Guide Sequences

L1 RNA expression can be inhibited using external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

### 5. ShRNA

L1 RNA expression can be inhibited using small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides (Brummelkamp et al., Science 296:550-553 (2002); Lee et al., Nature Biotechnol. 20:500-505 (2002); Miyagishi and Taira, Nature Biotechnol. 20:497-500 (2002); Paddison et al., Genes Dev. 16:948-958 (2002); Paul et al., Nature Biotechnol. 20:505-508 (2002); Sui (2002) supra; Yu et al., Proc. Natl. Acad. Sci. USA 99 (9): 6047-6052 (2002).

### B. Formulations

Provided herein are formulations for inhibiting L1 RNA. The NAs, small molecules and peptides described herein can be formulated for parenteral administration, parenteral administration or topical administration to the skin. The disclosed nucleic acids, small molecules and peptides can be administered to the skin using dosage forms and methods for delivering therapeutic agents and nucleic acids to the skin, in effective amounts to inhibit L1 RNA in the skin. In certain embodiments, the formulations include one or more cell penetration agents, e.g., transfection agents. The NA agent is mixed or admixed with a transfection agent (or mixture thereof) and the resulting mixture is employed to transfect cells. Preferred transfection agents are cationic lipid compositions, particularly monovalent and polyvalent cationic lipid compositions, more particularly LIPOFECTIN®, LIPOFECTACE®, LIPOFECTAMINE™, CELLFECTIN®, DMRIE-C, DMRIE, DOTAP, DOSPA, and DOSPER, and dendrimer compositions, particularly G5-G10 dendrimers, including dense star dendrimers, PAMAM dendrimers, grafted dendrimers, and dendrimers known as dendrigrafts and SUPERFECT®.

i. Parenteral Formulations

The compounds described herein (i.e., L1 RNA, vectors encoding L1 RNA, L1RNA-inhibiting NAs (or vectors encoding the same) and L1 RNA inhibiting agents) can be formulated for parenteral administration.

For example, parenteral administration may include administration to a patient intravenously, intradermally, intraperitoneally, intralesionally, intramuscularly, subcutaneously, by injection, by infusion, etc.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers. Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### 1. Controlled Release Formulations

The parenteral formulations described herein can be formulated for controlled release including immediate release, delayed release, extended release, pulsatile release, and combinations thereof.

a. Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contains two or more agents, the agents can be formulated for the same type of controlled release (e.g., delayed, extended, immediate, or pulsatile) or the agents can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the agent(s) is controlled by diffusion of the agent(s) out of the microparticles and/or degradation of the polymeric particles by hydrolysis and/or enzymatic degradation. Suitable polymers include ethylcellulose and other natural or synthetic cellulose derivatives. Like DNA and mRNA, siRNA and miRNA can be delivered via nanocarriers. For example, Benoit et al. Biomacromolecules. 2012; 1311:3841-3849 developed a di-block co-polymer (pDMAEMA-b-p(DMAEMA-co-PAA-co-BMA)) consisting of an siRNA complexation block (pDMAEMA) and an endosomal escape block (tercopolymer of PAA, BMA, and DMAEMA) for efficient siRNA delivery.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide)(PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the agent(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of agent containing microparticles. Additionally, proteins, polysaccharides and combinations thereof, which are water-soluble, can be formulated with agent into microparticles and subsequently cross-linked to form an insoluble network. For example, cyclodextrins can be complexed with individual agent molecules and subsequently cross-linked.

### 2. Method of Making Nano- and Microparticles

Encapsulation or incorporation of agent into carrier materials to produce agent-containing microparticles can be achieved through known pharmaceutical formulation techniques. In the case of formulation in fats, waxes or wax-like materials, the carrier material is typically heated above its melting temperature and the agent is added to form a mixture comprising agent particles suspended in the carrier material, agent dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, agent is added, and the molten wax-agent mixture is congealed under constant stirring as the mixture cools. Alternatively, the molten wax-agent mixture can be extruded and spheronized to form pellets or beads. These processes are known in the art. For some carrier materials it may be desirable to use a solvent evaporation technique to produce agent-containing microparticles. In this case agent and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, agent in a particulate form is homogeneously dispersed in a water-insoluble or slowly water soluble material. To minimize the size of the agent particles within the composition, the agent powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### 3. Injectable/Implantable Formulations

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods. Polyamides for nucleic acid delivery are described in U.S. Pat. No. 8,236,280

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

ii. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### 1. Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the agent and a controlled release polymer or matrix. Alternatively, the agent particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

### A. Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the agent with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT T®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT T® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired agent release profile can be achieved by combining low permeable and high permeable coating materials in suitable proportion.

The devices with different agent release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the agent is mixed with a wax material and either spray-congealed or congealed and screened and processed.

### B. Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating an agent or an agent-containing composition with a selected coating material. The agent-containing composition may be, e.g., a tablet for incorporation into a capsule, a tablet for use as an inner core in a “coated core” dosage form, or a plurality of agent-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

iii. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The formulations can include known excipients used in topical formulations, included but not limited to sunscreens, surfactants, preservatives, desquamation agents, antiperspirants, colorants, thickeners, skin lighteners, vitamins and other therapeutically active agents in a cosmetically acceptable carrier. The compositions may further contain one or more chemical penetration enhancers, membrane permeability agents, membrane transport agents, emollients, surfactants, stabilizers, buffers, and combination thereof.

“Penetration enhancers” are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Topical nucleic acid delivery using topical application, for example, topical application of naked DNA, DNA/liposomes or emulsion complex, liposomal cream, as well as physical methods such as stripping, electroporation, and micromechanical disruption methods

Methods of delivering nucleic acids (NAs) to the skin are known in the art. Physical methods include microneedle injection, microporation, electroporation, iontophoresis, sonophoresis, or passive delivery using polymeric nanoparticles; liposomes; peptides; or dendrimers. (Reviewed in Zakrewsky, et al., J. Control Release, 219:445-456 (2015)).

Intradermal injections are the simplest and most direct method for delivering NAs into the skin. Here, the barrier properties of the SC are overcome completely by injecting NAs directly into the viable tissue layers of the skin. Useful intradermal needles include microneedle arrays. Microneedle arrays comprise needles that are only 100-700 μm in length. When placed on the skin, their sharp tips allow easy insertion into the stratum corneum, while the short length ensures adequate penetration into the skin without disrupting nerves in deeper skin tissue. Microneedles can be used for delivery of nucleic acids disclosed herein, for example, plasmid DNA encoding L1 RNA, cationic lipid-DNA complexes (˜100 nm diameter), siRNA, etc.

Microporation is another technique that employs physical disruption of the SC (statun corneum) for delivery of large therapeutics or therapeutic carriers. An array of resistive elements can be placed on the skin. An electric current pulsed through the array results in localized ablation of corneocytes in contact with the array. Alternatively, erbium: yttrium-aluminum-garnet (Er:YAG) laser arrays can be used for localized ablation of the SC and epidermis. This techniques has been used to successfully deliver plasmid DNA, CpG oligonucleotides, siRNA, etc., to the skin.

Electroporation can be used to permeabilize the skin and enhance passive diffusion of agent. The mechanism of electroporation is quite different from that of electrically-induced microporation. Electrically-induced microporation utilizes electric fields to induce thermal ablation of SC microstructure creating pores in the skin. On the other hand, electroporation is the application of short duration (<0.5 s) and high intensity (<100 V) electric pulses to the skin which result in transient permeabilization of the lipid bilayers in the skin and concurrently permeabilize cell membranes of epidermal keratinocytes. Electroporation is also expected to create aqueous pores through the skin. Efficient delivery of nucleic acid molecules into skin by combined use of microneedle roller and flexible interdigitated electroporation array is disclosed in Huang, et al., Theranostics 2018; 8 (9): 2361-2376.

Iontophoresis can be used to drive transport of charged drugs like NAs. Applying a continuous low intensity (<10 V) electric field at a constant current.

Liposomes have also been studied extensively for nucleic acid delivery for the treatment of skin disease.

Highly ordered spherical complexes of nucleic acids (spherical nucleic acids) have shown potential for treating skin disease due to their enhanced delivery into skin, internalization into skin cells, and protection of NAs from degradation. Specifically, gold nanoparticles coated with a dense layer of highly-ordered and covalently bound siRNA resulted in passive transport through intact mouse SC and localized exclusively in the dermis and epidermis.

The formulations can include known skin penetration enhancers. Several peptides have been identified which possess the ability to enhance transport of NAs into the skin and elicit a therapeutic response. The first of these peptides discovered using phage-display screening was TD-1 (ACSSSPSKHCG)(SEQ ID NO:55). Hsu and Mitragotri identified another peptide using phage-display screening, SPACE peptide (ACTGSTQHQCG) (SEQ ID NO:56), with the ability to not only enhance delivery of siRNA across the skin but also enhance intracellular uptake (Hsu T, Mitragotri S Proc Natl Acad Sci USA. 2011 108 (38): 15816-21).

The present invention will be further understood by means of the following non-limiting examples.

### EXAMPLES

### I. Line-1 Retrotransposon RNA Delivery to Osteoporotic Patients Derived Mesenchymal Stem Cells Stimulates Osteogenic Differentiation and Bone Matrix Production

### Materials and Methods

### Participants

From 103 Norwegian women (50-86 years) with highly variable BMD, 30 were selected according to age, weight and key serum parameters and divided into two groups: Osteoporotic (T-score≤−2.5) and healthy (Tscore>−1). The procedures for patient registration, transiliacal bone biopsies and blood sampling were described previously. (47)(48) The participants were recruited through advertisements in newspapers and/or included via Lovisenberg Diaconal Hospital outpatient clinic. The study was approved by the Norwegian Regional Ethical Committee (REK no: 2010/2539) and conducted according to the Declaration of Helsinki.

### Human MSC Differentiation

Bone marrow-derived MSC (#C-12974, PromoCell GmbH, Heidelberg, Germany) were grown in 0.1% gelatin solution (#07903, StemCell)-coated plates until passage 4. Growth medium (#PT-3001, Lonza) was changed every 3 days. Osteogenic differentiation was induced by replacing growth medium with Osteogenic Differentiation Medium (#PT-3002, Lonza) on 70% confluent cells seeded on 1:50 Matrigel (#356237, Corning)-coated plates. Adipogenic differentiation was induced by three cycles of induction/maintenance Adipogenic Differentiation Medium (#PT-3004, Lonza) on post-confluent cells seeded on 1:50 MatMatrigel (#356237, Corning)-coated plates.

Genomic DNA Extraction and TaqMan qPCR-Based L1 CNV Assay

High molecular weight genomic DNA (HMW-gDNA) was isolated using MagAttract HMW DNA Kit (#67563, Qiagen), following the manufacturer's instructions. During lysis, the samples were treated with RNase H and proteinase K (both provided in the kit) for at least 1 hour at 37° C. to remove RNA/DNA substrates and protein contamination, respectively. Isolated HMW-gDNA was finally treated with Exonuclease I (#M0568, NEB) for 30 min at 37° C. and then deactivated for 15 min at 80° C. to remove free ssDNA. HMW-gDNA was then analyzed for L1 copy number using a 7900HT Fast Real Time PCR (Applied Biosystems). All copy number assays for L1 were normalized on human centromeric alpha satellite (SATA) as repetitive endogenous control for DNA input concentration. Each sample was analyzed in triplicate. For each reaction, a 20 μl mix of gDNA (25 pg), target specific primers (0.2 μM), target specific FAM-labeled probe (0.4 μM), ROX passive reference dye (0.4 μl, #1725858, Bio-Rad) and IQ Multiplex Powermix (10 μl, #1725849, Bio-Rad) was incubated at 95° C. for 3 minutes, followed by 40 cycles of denaturation at 95° C. for 45 seconds and primer annealing/extension at 59° C. for 45 seconds. TaqMan probes and primers sequences for active, retrotransposition-competent, L1 used for CNV study are published (Coufal, et al. Nature (2009), doi: 10.1038/nature08248; Goodier, et al. DNA (2014), doi: 10.1186/1759-8753-5-11) and shown below, and are the primers and probes used in this study.

L1 5′UTR-ORF1
Forward primer:
(SEQ ID NO: 2)
5′-GAATGATTTTGACGAGCTGAGAGAA-3′;
Reverse primer:
(SEQ ID NO: 3)
5′-GTCCTCCCGTAGCTCAGAGTAATT-3′;
Probe sequence:
(SEQ ID NO: 4)
5′-AAGGCTTCAGACGATC-3′^{(30, 37)};
L1 ORF2
Forward primer:
(SEQ ID NO: 5)
5′-TGCGGAGAAATAGGAACACTTTT-3′;
Reverse primer:
(SEQ ID NO: 6)
5′-TGAGGAATCGCCACACTGACT-3′;
Probe sequence:
(SEQ ID NO: 7)
5′-CTGTAAACTAGTTCAACCATT-3′^{(30, 37)}.
SATA
Forward primer:
(SEQ ID NO: 8)
5′-GGTCAATGGCAGAAAAGGAAAT-3′;
Reverse primer:
(SEQ ID NO: 9)
5′-CGCAGTTTGTGGGAATGATTC-3′;
Probe sequence:
(SEQ ID NO: 10)
5′-TCTTCGTTTCAAAACTAG-3′^{(30, 37)};
RPL13A
Forward primer:
(SEQ ID NO: 11)
5′-GAAAGCCAAGATCCACTACC-3′;
Reverse primer:
(SEQ ID NO: 12)
5′-TGGGTCTTGAGGACCTCTGT-3′;
RUNX2
Forward primer:
(SEQ ID NO: 13)
5′-TCAACGATCTGAGATTTGTGGG-3′;
Reverse primer:
(SEQ ID NO: 14)
5′-GGGGAGGATTTGTGAAGACGG-3′;
OCN
Forward primer:
(SEQ ID NO: 15)
5′-GGCGCTACCTGTATCAATGG-3′;
Reverse primer:
(SEQ ID NO: 16)
5′-GTGGTCAGCCAACTCGTCA-3′;
OPN
Forward primer:
(SEQ ID NO: 17)
5′-GAAGTTTCGCAGACCTGACAT-3′;
Reverse primer:
(SEQ ID NO: 18)
5′-GTATGCACCATTCAACTCCTCG-3′;
BSP
Forward primer:
(SEQ ID NO: 19)
CACTGGAGCCAATGCAGAAGA;
Reverse primer:
(SEQ ID NO: 20)
5′-TGGTGGGGTTGTAGGTTCAAA-3′;
OSX
Forward primer:
(SEQ ID NO: 21)
5′-CCTCTGCGGGACTCAACAAC-3′;
Reverse primer:
(SEQ ID NO: 22)
5′-AGCCCATTAGTGCTTGTAAAGG-3′;
TBP
Forward primer:
(SEQ ID NO: 23)
5′-GCTGGCCCATAGTGATCTTT-3′;
Reverse primer:
(SEQ ID NO: 24)
5′-CTTCACACGCCAAGAAACAGT-3′;
PPARγ
Forward primer:
(SEQ ID NO: 25)
5′-ACCAAAGTGCAATCAAAGTGGA-3′;
Reverse primer:
(SEQ ID NO: 26)
5′-ATGAGGGAGTTGGAAGGCTCT-3′;
FABP4
Forward primer:
(SEQ ID NO: 27)
5′-ACTGGGCCAGGAATTTGACG-3′;
Reverse primer:
(SEQ ID NO: 28)
5′-CTCGTGGAAGTGACGCCTT-3′;
FASN
Forward primer:
(SEQ ID NO: 29)
5′-AAGGACCTGTCTAGGTTTGATGC-3′;
Reverse primer:
(SEQ ID NO: 30)
5′-TGGCTTCATAGGTGACTTCCA-3′;
LPL
Forward primer:
(SEQ ID NO: 31)
5′-AGGATGTGGCCCGGTTTATC-3′;
Reverse primer:
(SEQ ID NO: 32)
5′-CCAAGGCTGTATCCCAAGAGAT-3′;
GFP-968/1013
Forward primer:
(SEQ ID NO: 33)
5′-GCACCATCTTCTTCAAGGACGAC-3′;
Reverse primer:
(SEQ ID NO: 34)
5′-TCTTTGCTCAGGGCGGACTG-3′;

L1 TaqMan primers and probes specificity analysis was performed: L1-5′-ORF1 primers and probe set matches 309 sequences (246 L1HS-Ta1; 1 L1HS-Ta0; 1 L1HS-preTa; 61 L1PA2), L1-ORF2 primers and probe set matches 181 sequences (161 L1HS-Ta1; 3 L1-HS-Ta0; 4 L1HS-preTa; 6 L1PA2; 1 L1PA3; 5 L1PA4).

### Lamivudine 3TC Treatment

Lamivudine 3TC (#L1295, Sigma) was resuspended in DMSO and added to cell medium every 24 hours in a 150 μM final concentration.

### Mineralization Assay

Cells were washed in PBS and fixed with 4% Paraformaldehyde for 15 minutes. Mineralization was assessed by using the OsteoImage Mineralization Assay (#LOPA503, Lonza) according to manufacturer's indication. Mineralization was quantitatively assayed with GloMax discover plate reader (Promega) with appropriate excitation (492)/emission (520) wavelengths.

### Lipid Content Assay

Cells were washed once in PBS and incubated for 10 minutes with AdipoRed Assay reagent (#LOPT7009, Lonza). Lipid content was quantitatively assayed with GloMax discover plate reader (Promega) with appropriate excitation (485)/emission (572) wavelengths.

RNA Extraction and cDNA Preparation

Cells were harvested and resuspended in 1 ml of QIAzol Lysis reagent (#79306, Qiagen). Total RNA was then purified with RNeasy Plus Mini kit (#74134, Qiagen) with minimal modifications to manufacturer's instructions. DNase treatment (RNase free DNase set, #79254, Qiagen) was performed to remove any residual DNA. RNA quality and concentration were checked using a Nanodrop 2000 spectrophotometer (ThermoFisher). cDNA was synthesized from 200 ng of each RNA sample using a Superscript III first-strand cDNA synthesis system (#18080051, ThermoFisher) according to manufacturer's protocol.

L1 RNA transfection

The vector human-L1_pBluescript II sk (+) carrying the full length L1 sequence was custom-prepared by GenScript, USA. Large-scale human L1 mRNA was in vitro transcribed, modified and purified by TriLink Biotechnologies, USA, (ARCA capped and 2′Omethymalted (CapI), fully substituted with 5-methyl-C, 25% substitution of Cyanine-5-U and 75% substitution of Pseudo-U, enzymatically polyadenylated, DNase and phosphatase treated, silica membrane purified). L1 RNA was transfected in differentiating osteoblasts at day 7 using Lipofectamine™ MessengerMAX™ (Invitrogen, USA, Cat. No. LMRNA003) with a modified protocol were a lower RNA amount (10 times less) than recommended was transfected. RFP mRNA (System Bioscience, USA, Cat. No. MR800A-1) was used as negative control. 3 days after transfection, bone matrix was quantified with OsteoImage Mineralization Assay (Lonza, Basel, Switzerland, Cat. No. LOPA503).

### Alizarin Red Staining

Osteoblasts were washed with 1×PBS (Kantonsapotheke Zürich, Switzerland, Cat. No. A171012) and fixed with 4% (v/v) formaldehyde (Sigma, USA, Cat. No. F8775) in 1×PBS for 30 min. After washing twice with ddH2O, Alizarin Red staining solution (0.7 g Alizarin Red S (Sigma, USA, Cat. No. A5533) diluted in 50 ml ddH2O at pH=4.2) was added for 20 min. Afterwards, cells were washed four times with ddH2O, dried, and stored in the dark until image acquisition. For absorbance measurement, Alizarin Red S was eluted from stained osteoblasts with 300 μl 10% (w/v) cetylpyridinium chloride in an aqueous 0.01 M Na2HPO4/NaH2PO4 solution at pH=7 for 1 h. One hundred fifty microliters were transferred on a 96-well plate, and absorbance was measured at 560 nm. Ten percent (w/v) cetylpyridinium chloride in an aqueous 0.01 M Na2HPO4/NaH2PO4 solution was used as blank. Images were acquired, processed and analysed as previously described (Eggerschwiler et al., Stem Cell Res. Ther. (2019). doi: 10.1186/s13287-019-1170-8).

### Gene Expression Analysis in Differentiating Osteoblasts and Adipocytes

Real time quantitative polymerase chain reaction (qPCR) was performed with 7900HT Fast Real Time PCR system (Applied Biosystems). Each sample was analyzed in triplicate and normalized with the endogenous control, Ribosomal Protein L13A (RPL13A) for osteogenesis and Tata Binding Protein (TBP) for adipogenesis, for cDNA input concentration. No template and no RT were included as negative controls. For each 15 μl reaction, 10 ng (1 ng for L1) of cDNA was mixed with 1 μM specific primers mix and 7.5 μl of Sybr Select Master mix (#4472908, Life Technologies). The reaction was incubated at 95° C. for 10 minutes, followed by 40 cycles of denaturation at 95° C. for 15 seconds, annealing at 60° C. for 30 seconds and elongation at 72° C. for 30 seconds. Ct values were calculated by 7900HT Fast Real Time PCR RQ manager software (Applied Biosystems) and then normalized as DCt between the gene of interest and the endogenous calibrator. Primers used in this study for gene expression analysis were designed using Primer3 (http://www.ncbi.nlm.nih.gov/tools/primer-blast/). In all primer pairs each primer matches a different exon. Amplicons length was 80-130 nucleotides. Primers sequences are reported in Table 1.

### In Vitro Retrotransposition Assay

150×10³ MSC were incubated with 3 μg of LRE3-EGFP plasmid (kindly provided by Prof. Fred Gage) and electroporated with Neon transfection system (ThermoFisher). Cells were subjected to one pulse of 990V for 40 ms, recovered for 48 hours and then induced to differentiate into mature osteoblasts for two weeks. Cells were harvest and DNA was isolated. 50 ng of DNA was used as template to amplify the EGFP sequence with intron flanking oligos to discriminate between the intron containing RC-L1 sequence carried by the plasmid (1243 bp, not retrotransposed) and the spliced newly inserted one (343 bp, retrotransposed). PCR reaction was performed with 0.5 μM of each primer and 10 μl of Hot start premix Taq DNA polymerase (#R028A, Takara) in a final volume of 20 μl and incubated at 94° C. for 30 seconds for denaturation, at 58° C. for 30 seconds for primers annealing and at 72° C. for 1 minute for primers extension. The cycle was repeated 30 times. GFP primers sequences are published (38) and reported in Table 1.

### Cell Cycle Analysis

2×105 MSCs were trypsinized for 5 min at 37° C., washed with PBS and 2% BSA, passed through a 70 μM strainer (#352350, Corning) and then fixed at −20° C. for 30 min in 70% ethanol. After washing with PBS and 4% BSA, cells were resuspended in PBS and incubated 1 hour at 37° C. with RNAse. Cells were then washed and resuspended in 100 μl of Flow Cytometry Staining Buffer (R&D System, #FC001). 10 μl of 1 mg/ml Propidium iodide (PI) staining solution (#P3566) was added to the single cell solution, gently mixed, and incubated 5 min in the dark. Cell cycle analysis was performed on BD FACSCanto II Flow Cytometry System, using BD FACSDiva Software.

### Antisense Oligonucleotides Delivery

For L1 knock-down experiments, FANA (2-deoxy-2-fluoroarabinonucleic acids) modified ASOs specific for 5 different LINE-1 ORF1 RNA regions, and one scrambled (SCR) used as negative control, were delivered by gymnosis according to producer's instructions (AUMbiotech). Lyophilized oligonucleotides were resuspended in Nuclease free water at a concentration of 500 μM and then diluted to 5 μM in cell medium every three days.

### Statistical Analysis

To determine the significance between two mean values, comparisons were made by appropriated Student's t test where the 0.05 level of confidence was accepted for statistical significance. *=P-value<0.05; **=P value<0.005; ***=P-value<0.0005; ****=P-value<0.00005. In correlation analysis, p-value and coefficient of determination (R-squared, R2) were calculated using GraphPad (https://www.graphpad.com/quickcalcs/). The number of biological replicates (N) is indicated in the plots or in figure legends.

### Results

### L1 DNA Copy Number is Expanded in the Genome of Healthy Bone

Variation in the copy number of active, retrotransposition-competent, L1, was analyzed in genomic DNA of 30 transiliac biopsies from age matched postmenopausal women classified as healthy (CTR, n=14, BMD t-score≥−1) or osteoporotic (OP, n=16, BMD t-score<−2.5)(data not shown). In brief: all donors were on standard Norwegian diet and had similar nutritional supplements and life style factors, including physical engagement. They had normal endocrine, clinical, biochemical and nutritional status and had been postmenopausal without estrogen medication for at least 2 years. They received no drug treatment known to affect bone turnover and were free of other skeletal primary or secondary diseases. No resorption markers (serum TRAP5B, 1CTP, urine NTX or urine DPD) differed between patients and healthy, and they were all within normal laboratory ranges according to international standards. Of bone formation markers, serum osteocalcin was in normal range and did not differ between groups while bone specific alkaline phosphatase (ALP), although within normal variation, was significantly elevated in osteoporosis (p<0.019).

To estimate variations in L1 genomic copy number TaqMan qPCR coupled to isolation of high molecular weight genomic DNA, ssDNA (e.g. reversed transcribed but not integrated L1 cDNA) and RNA/DNA substrates removal by Exonuclease I and RNase H treatment respectively, as reported in the methods, was used. This procedure is state of the art to exclude the detection of L1 sequences that are not integrated into the genome and, therefore, to avoid overestimation of their genomic copy number as previously anticipated (Goodier, et al. DNA (2014), doi: 10.1186/1759-8753-5-11; Goodier, et al. DNA (2016), doi: 10.1186/s13100-016-0070-z) and recently reported (34). Using TaqMan qPCR, two different regions of L1 DNA sequence (5′UTR-ORF1 and ORF2) were amplified in copy number variation (CNV) assays.

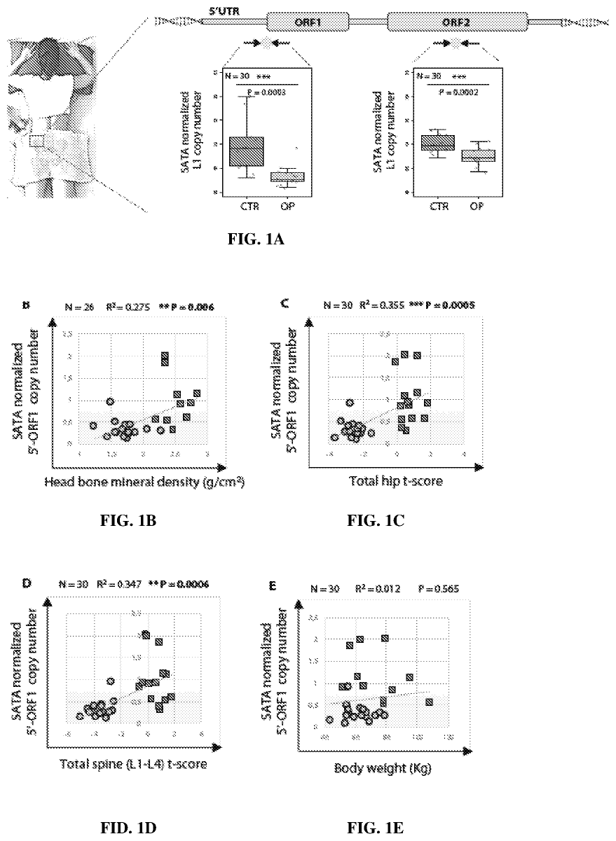

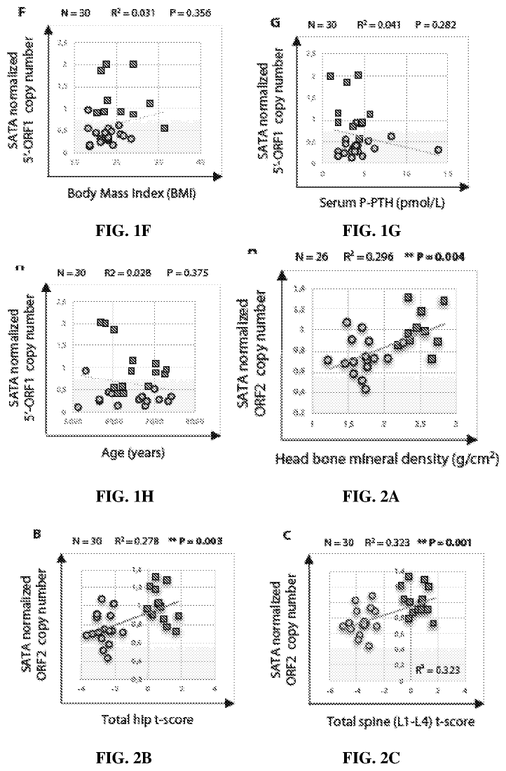

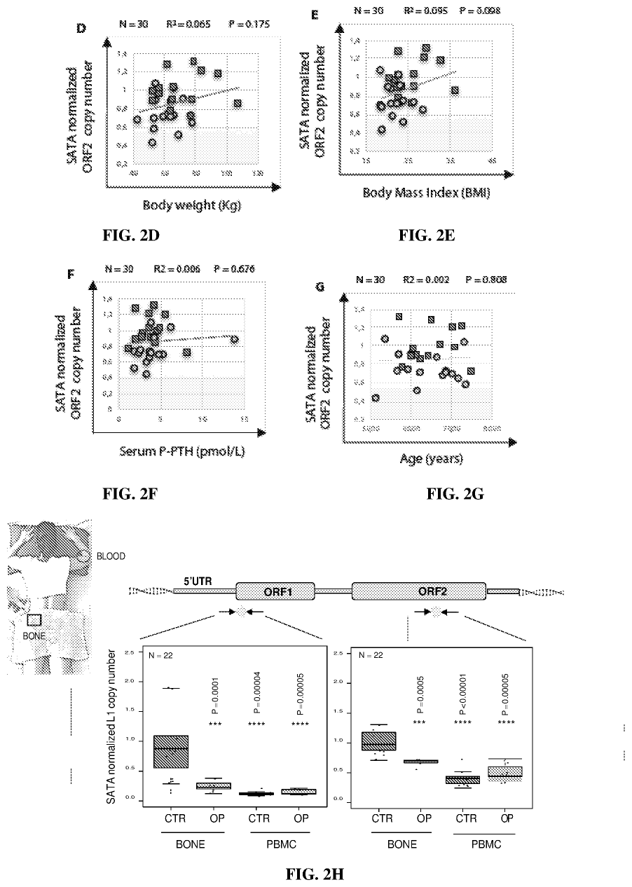

The variation of L1 copy number between the two groups was highly significant for both of the sequences, showing a strong reduction in patients (FIG. 1A). Two sets of validated TaqMan primers and probes specific for potentially active, retrotranspositions-competent L1 (see methods for TaqMan primers and probes specificity analysis) were used (Coufal, et al. Nature (2009), doi: 10.1038/nature08248; Muotri, et al. Nature (2010), doi: 10.1038/nature09544). Consistently, the observed relative variation in L1 copy number between healthy and patients represents a difference limited to the small portion of potentially active L1HS-Ta1 family. The data shows that L1 5′UTR-ORF1 copy number in bone genome correlates positively with BMD at all sites measured: head (R2-0.275; p=0.006)(FIG. 1B), total hip (R2=0.355; p=0.0005)(FIG. 1C), and spine (R2=0.347; p=0.0006) FIG. 1D). In contrast, no statistically significant correlation was observed with individual parameters not strictly related to skeleton metabolism, such as body weight (R2=0.012; p=0.565)(FIG. 1E), body mass index (R2=0.031; p=0.356)(FIG. 1F) parathyroid hormone level in the serum (R2=0.041; p=0.282)(FIG. 1G) and age (R2=0.028; p=0.375)(FIG. 1H). Consistent results were obtained when clinical parameters were correlated to L1 ORF2 copy number using defined primers and probes (FIG. 2A-G).

To assess whether the variation in L1 copy number between CTR and OP women was specific for bone tissue, copy number variation (CNV) assay was performed on the genome of peripheral blood mononuclear cells (PBMC) taken from the same donors. In PBMC genome, L1 copy number was markedly lower than in healthy bone but, most importantly, no variation was observed between CTR and OP groups (FIG. 2H). These results suggest that when comparing osteoporotic patients with healthy donors, quantitative variation in L1 genomic copy number is detected specifically in bone and not in other mesoderm-derived tissues not affected by the pathology, demonstrating the bone specificity of L1 dynamics alteration in osteoporosis.

### The Osteogenic Differentiation of MSC Triggers L1 Genomic Expansion

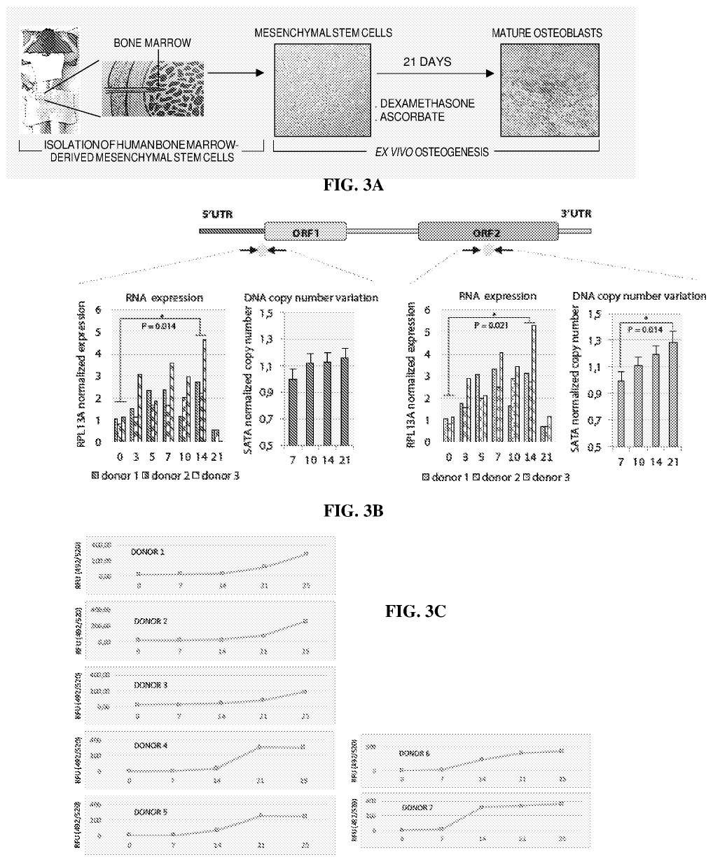

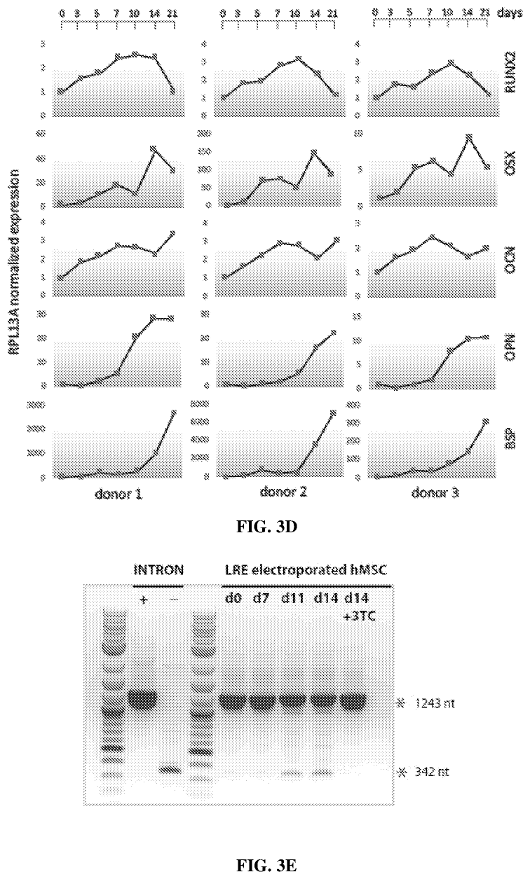

The genetic cause of osteoporosis is unknown, but it is associated with a faulty differentiation of MSC toward the osteogenic lineage in the bone marrow niche. The observed reduced number of L1 copies in the genome of postmenopausal osteoporotic bone indicated a potential association between L1 mobilization and bone development and that a failure in L1 reactivation could be involved in defective bone formation. Therefore, additional studies investigated whether L1 retrotransposons activation and expansion does occur during physiological osteogenesis of adult MSC. Bone marrow-derived MSC isolated from the iliac bone of healthy donors were differentiated into mature osteoblasts for three weeks (FIG. 3A). Bone-like nodules deposited by mature osteoblasts were detected with optical microscope (FIG. 3A). The increase in calcified matrix deposition and osteogenic genes expression indicated that bone differentiation occurred successfully ex vivo (data not shown, and FIG. 3C-D). Moreover, as the onset of mineralization may differ between MSC donors, age-matched donors were chosen (about same age as the cohorts studied) presenting similar mineralization dynamics (FIG. 3C) as well as similar marker genes expression (FIG. 3D), in order to ensure a consistent behavior of the cell system. First, Real Time qPCR was used to monitor the timeline of L1 expression in developing osteoblasts and found that soon after osteogenic induction, the intracellular levels of L1 RNA gradually increased, and then decreased at the end of differentiation (FIG. 3B). Using TaqMan qPCR-based CNV assay on HMW-gDNA, studies investigated whether the differentiation-induced L1 expression was accompanied by changes in L1 de novo genomic integrations. As shown in FIG. 3B, L1 copy number increased significantly in mature osteoblasts (day 21) compared to undifferentiated cells (day 7). An engineered L1 retrotransposition GFP-reporter based assay, previously used in several studies (Coufal, et al. Nature (2009), doi: 10.1038/nature08248; Ostertag, et al. Nucleic Acids Res (2000), doi: gkd248 [pii]; Macla, et al. Genome Res. (2017), doi: 10.1101/gr.206805.116), confirmed that L1 propagation accompanies bone cells differentiation (FIG. 3E).

### Impairment of L1 Dynamics is Detrimental for Osteoblasts Maturation

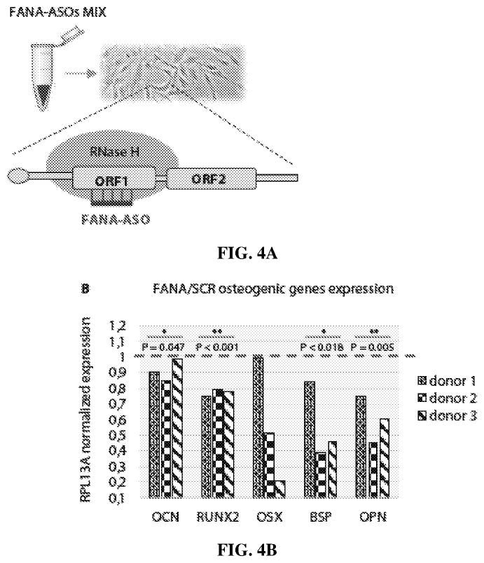

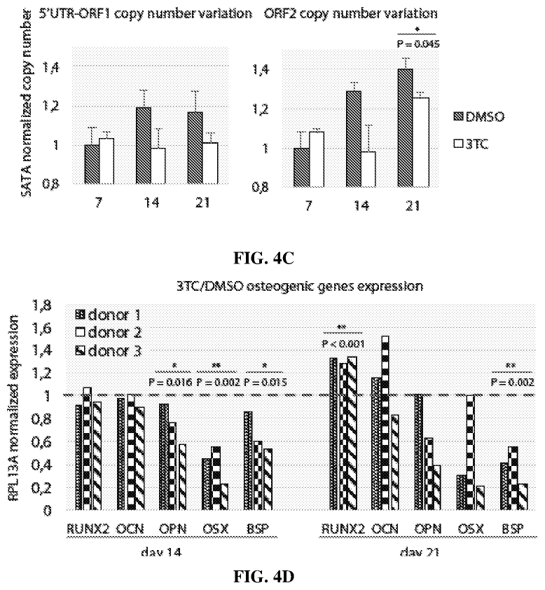

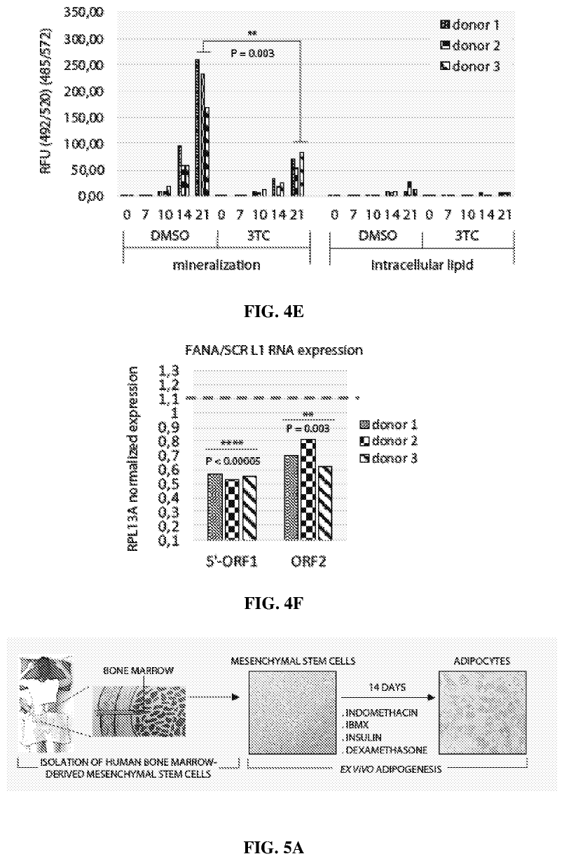

To understand whether L1 reactivation and genomic expansion in developing osteoblasts affect the osteogenic phenotype, L1 RNA was knocked down by using fluoroarabinonucleic acids (FANA) modified ASOs specific for L1-ORF1 RNA sequence. FANA ASOs bind the target sequence and act as docking elements for RNAseH-mediated cleavage (FIG. 4A), thereby avoiding any off-target effects of RNA-induced silencing complex (RISC). Importantly, L1 sequences are frequently present in the introns of genes and, consequently, in the nuclear precursors of many RNAs that possibly may become targets of anti-L1 ASOs. The ASOs that were used for knocking-down L1 RNAs are mostly excluded from the nucleus of the cells (data not shown). This further reduces the possibility of off-targeting. A mix of five FANA ASOs was delivered every three days to differentiating osteoblasts and analyzed the expression of bone related genes by Real Time qPCR. Somewhat surprisingly, a moderate depletion of L1 RNA (FIG. 4F) was sufficient to induce a significant reduction in the expression of the osteoblasts-related transcription factors Osterix (OSX, −43%) and Runt-related transcription factor 2 (RUNX2, −23%), analyzed 16 days after the induction of bone formation. Moreover, similar observations were made for the osteoblasts-specific gene Osteocalcin (OCN or BGLAP, −10%) and two of the main non-collagenous components of bone tissue Osteopontin (OPN, −40%) and Bone Sialoprotein (BSP, −44%)(FIG. 4B). The results indicated that somatic L1 RNA depletion undermined the cells ability to activate osteogenic program and to produce mineralized bone.

### NRTI-Mediated Inhibition of L1 Genomic Expansion Reduces Maturation and Impairs Mineralization of Developing Osteoblasts

Further, initial studies investigated whether block of ORF2-mediated L1 retrotransposition by NRTI Lamivudine 3TC (3TC) preventing the expansion of L1 copy number in developing osteoblasts, was affecting bone cells maturation and function. Differentiating osteoblasts were treated with and without 3TC every day for three weeks, and L1 copy number was measured at three different time points of differentiation. As expected, the drug efficiently prevented L1 DNA expansion during osteoblasts maturation (FIG. 4C). To assess the possible phenotypic effects of 3TC on osteogenic markers, expression of marker genes with (3TC) and without (DMSO) 3TC-treatment was analyzed in differentiating osteoblasts. In terminally differentiated cells (day 21) a highly significant reduced expression of OPN (−23%), OSX (−50%) and BSP (−60%) was observed (FIG. 4D). Coherently, mineral matrix deposition was significantly reduced (−60%)(FIG. 4E). Potential detrimental effects of the drug on cell viability, were excluded by Propidium Iodide staining followed by cell cycle FACS analysis on 3TC treated osteoblasts. Results of FACS cell cycle analysis of human mesenchymal stem cells treated (3TC) or not treated (DMSO) with lamivudine 3TC and measurement of apoptotic cell number in sub-G1 peak showed no significant differences (data not shown). These results corroborate the hypothesis of L1 genomic expansion inhibition representing a tight link between NRTI treatment and mineralization loss in patients under ART.

MSC Differentiating into Adipocytes Lack L1 Mobilization

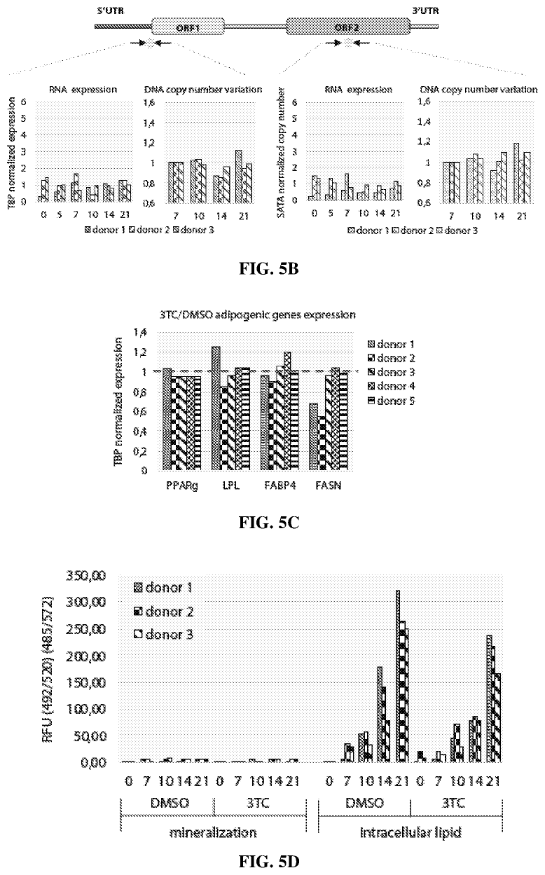

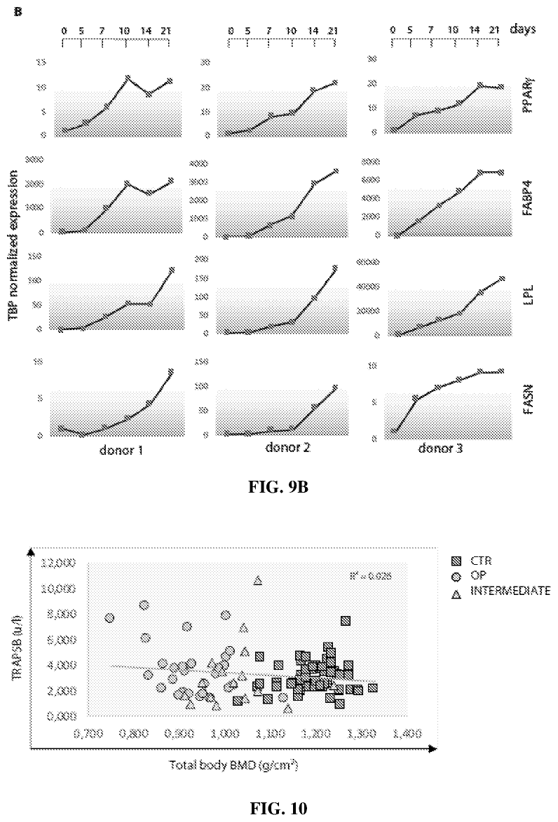

Our findings uniformly demonstrate that MSC are unable to efficiently differentiate into functional osteoblasts when L1 reactivation is inhibited. In postmenopausal osteoporosis red bone marrow changes from red to white as the fat content increases (Devlin, et al. Lancet Diabetes Endocrinol. (2015), doi: 10.1016/S2213-8587 (14) 70007-5; Ambrosi, et al. Cell Stem Cell (2017), doi: 10.1016/j.stem.2017.02.009). When mesodermal progenitors were differentiated ex vivo into fat cells (FIG. 5A) lipid droplets accumulated by adipocytes were easily detected with optical microscope (FIG. 5A). The increase of intracellular fatty acid content and adipogenic genes expression indicated that adipogenesis occurred successfully ex vivo (FIGS. 9A-B).

The expression and the copy number (FIG. 5B) of L1 was monitored without observing significant changes upon differentiation. This is consistent with a previous report demonstrating that MSCs differentiating into adipocytes are not competent for retrotransposition (Macla, et al. Genome Res. (2017), doi: 10.1101/gr.206805.116). Finally, as shown in five different donors, 3TCmediated inhibition of L1 expansion in MSC did not significantly affect adipogenic marker genes expression (FIG. 5C) and accumulation of intracellular lipid was unaltered (FIG. 5D). These studies led to a conclusion that in developing MSC somatic L1 reactivation appears to be lineage-specific and required for osteogenic program, while it is not involved in the formation of fat cells, the prevalent cell type in the marrow niche of osteoporotic patients.

### Line-1 Retrotransposon RNA Delivery to Mesenchymal Stem Cells Stimulates Osteogenic Differentiation and Bone Matrix Production

Bone L1 Copy Number Correlates with Mature Osteoblasts and Osteocytes Activity.

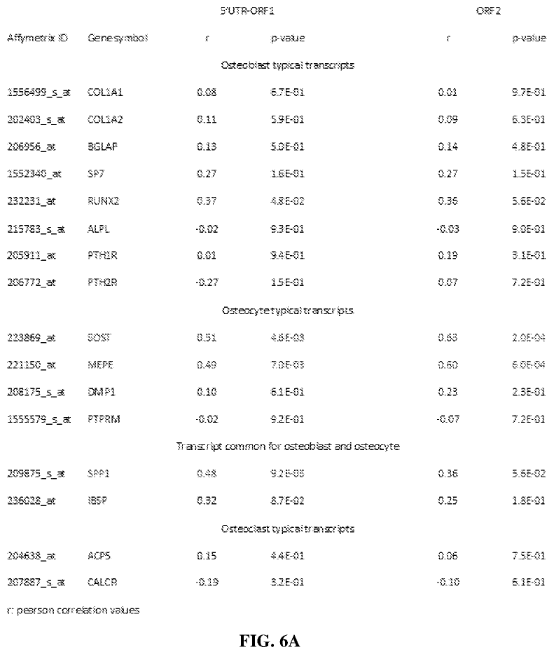

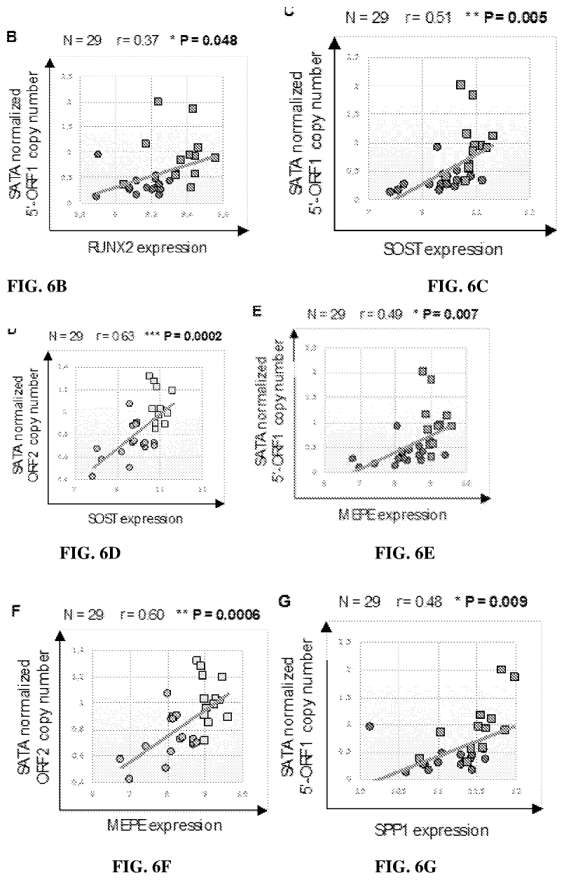

FIGS. 6A-F show the correlation between L1 copy numbers and the expression of osteoblast, osteocyte and osteoclast specific genes in the biopsies of the 30 selected participants. The RUNX2 transcript levels in osteoblasts were marginally significant for 5′UTR-ORF1 region (FIGS. 6A and 6B). Of four osteocyte markers, two were positively correlated with L1 copy number: SOST (p=0.005 for 5′UTR-ORF1; p=0.0002 for ORF2) (FIGS. 6A, C and D) and MEPE (p=0.007 for 5′UTR-ORF1; p=0.0006 for ORF2)(FIGS. 6A, E and F). Significant correlation was also found between SPP1, commonly expressed in both mature osteoblasts and osteocytes, and 5′UTR-ORF1 region copy number (p=0.009)(FIGS. 6A and G), but not for the osteoclast specific markers ACP5 and CALCR (FIG. 6A). These data strongly associate the reduced L1 copy number in bone of osteoporotic patients with the impaired anabolic activity of osteoblast/osteocyte within the same tissue.

Synthetic I1 RNA Delivery to MSC from Steoporotic Patients Triggers Matrix Mineralization in Culture

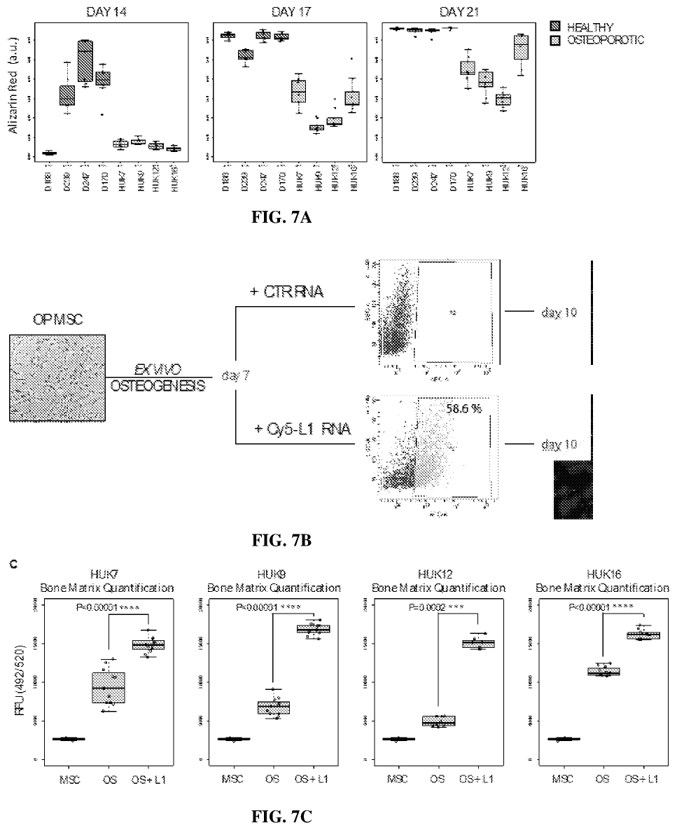

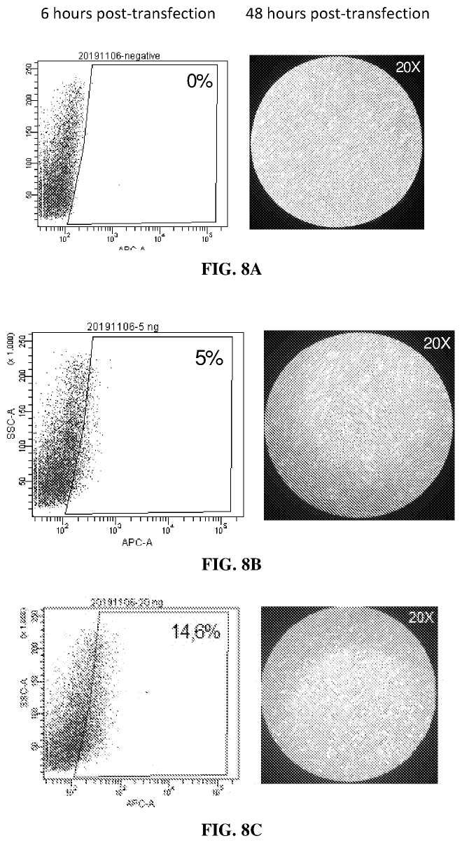

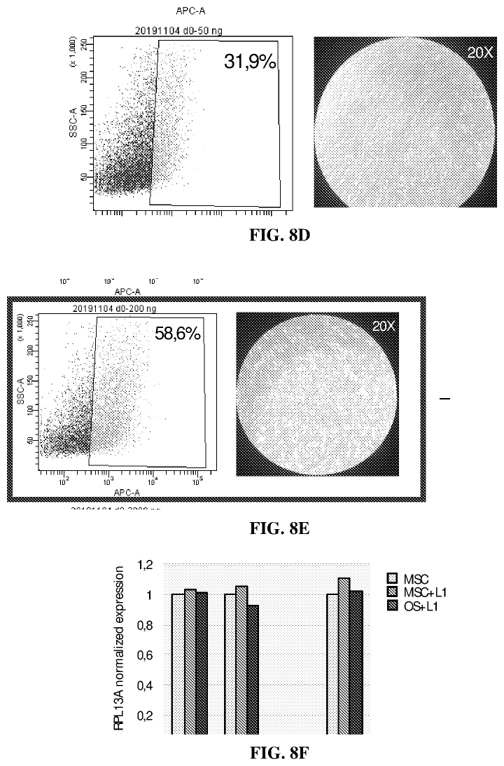

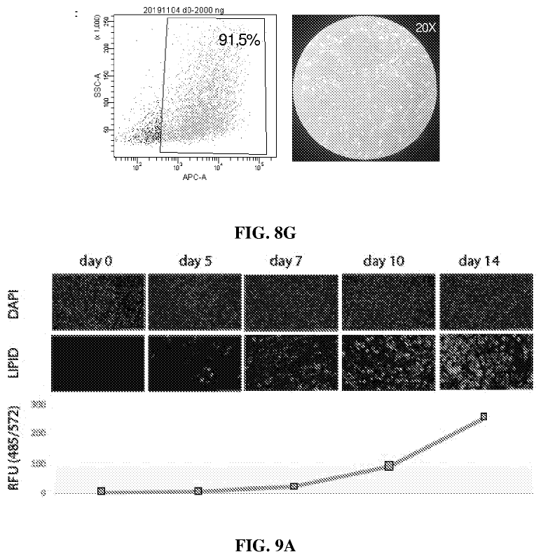

Osteoporotic bone shows a clear defect in L1 reactivation in vivo possibly with negative consequences for osteoblastic bone formation. Thus, additional studies sought to test if direct L1 RNA delivery to MSC differentiating to osteoblasts, obtained from osteoporotic donors, and could improve maturation and osteogenic capability. MSC were isolated from femur of four healthy donors and four patients and tested for their ability to support osteogenic differentiation (FIG. 7B). A low dose (FIG. 8A-G) of Cy5-conjugated synthetic full-length L1 RNA was transfected to these differentiating osteoblasts, with high efficiency (FIG. 7B). As expected, the exogenous lipofectamine-mediated RNA delivery resulted in the formation of intracellular vesicles (FIG. 7B, red foci) where L1 RNA was released from slowly over time (Kirschman, J. L. et al Nucleic Acids Res. 2017; doi: 10.1093/nar/gkx290). While cells from patients showed a markedly delayed and reduced mineralization (FIG. 7A), transfected cells with L1 RNA showed restored bone matrix production (FIG. 7C). Of note, capping, 2′-O-Methylation of 5′ end, polyadenylation (200 adenosines), full substitution with 5-methylcytidine (m5C) and 75% substitution with pseudouridine were used to stabilize RNA and to bypass the intracellular innate immune system (Koski et al., J. Immunol 2004; doi: 10.4049/jimmunol.172.7.3989; Pardi et al., Methods Mol. Biol. (2013). doi: 10.1007/978-1-62703-260-5_2; Ludwig, J. et al. Nat. Struct. Mol. Biol. (2010). doi: 10.1038/nsmb.1863; Karikó et al., Immunity (2005). doi: 10.1016/j.immuni.2005.06.008; Anderson, B. R. et al. Nucleic Acids Res. (2010). doi: 10.1093/nar/gkq347; Kormann, et al. Nat. Biotechnol. (2011) doi: 10.1038/nbt.1733).

Accordingly, neither apoptosis nor Interferon response genes were induced upon L1 RNA transfection (FIG. 8G). The results show that in all patients tested, the delivery of L1 RNA to in vitro differentiating MSC greatly enhanced osteoblasts maturation and fully rescued the production of mineralized matrix.

### DISCUSSION

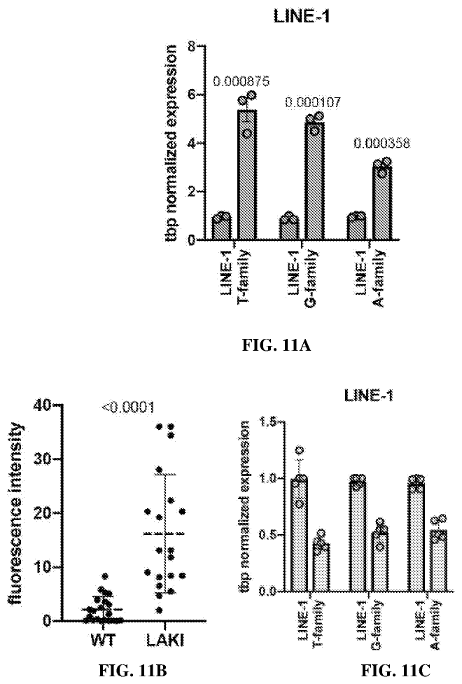

Primary osteoporosis is one of the most common and costly diseases worldwide in relation to societal expenses and human incapacitation (Cunningham, et al. Osteoporos. Int. (2016), doi: 10.1007/s00198-016-3620-9). Secondary osteoporosis frequently occurring due to other diseases, medication and insufficient nutrition may be even more frequent, but receives less attention, especially patients under NRTI-based antiretroviral treatment. These studies report L1 genomic structural variations are associated with bone density of 30 postmenopausal women, where higher amount of L1 DNA copies was observed in healthy compared to osteoporotic bone (FIG. 1A). Notably, this structural L1-driven genomic variation between CTR and OP women was specifically observed in bone, but not in peripheral blood (FIG. 2H), also representing cells of mesenchymal origin, and obtained from the same donors. This in vivo observation in well-defined postmenopausal healthy versus osteoporotic women suggested that the expansion of L1 elements may represent a genomic record of normal bone development and/or structural maintenance. Using mesenchymal stem cell progenitors from human marrow, adult bone formation ex vivo was recapitulated and the studies demonstrated a developmentally regulated reactivation and mobilization of L1 accompanying maturation of osteoblasts (FIG. 3A-B). The ASOs-mediated degradation of L1 RNA as well as the NRTI-mediated inhibition of L1 retrotransposition during bone formation severely affected osteoblasts maturation with deleterious impact on mineralization (FIG. 4A-E). The dramatic phenotypic effect was not due to general cellular toxic effects (FIG. 5A-B) but appear to be lineage and osteogenic developmental program specific as shown also by the lack of significant effects of L1 loss of function on adipogenesis (FIG. 5A-D). Notably, NRTI-mediated inhibition of L1 genomic expansion did not alter the expression of key adipogenic genes nor lipid accumulation in developing adipocytes. Our findings that reduced L1 activity in differentiating MSC leads to defective osteogenesis and reduced osteoblast dependent mineralization, but does not limit lipid accumulation in developing adipocytes, is coherent with the bone loss and the increased marrow fat tissue characterizing the primary osteoporosis disorder (Hawkes, et al. Bone (2018), doi: 10.1016/j.bone.2018.03.012). Moreover, it has been recently demonstrated that in vivo Lamivudine treatment increases marrow fat tissue in mice (Cecco, et al. Nature 566, 73-78 (2019). Our data are in accordance with a well-documented association between NRTI-based therapies and bone loss in patients (Grigsby, et al. Osteoporos. Int. (2004), doi: 10.1007/s00198-004-1627-0; Brown, et al. AIDS (2006), doi: 10.1097/QAD.0b013e32801022eb; Madeddu, et al, Q J Nucl Med Mol Imaging (2004), and with the fact that ORF2 is an established and recognized target of NRTIs (Jones, et al. PLoS One (2008), doi: 10.1371/journal.pone.0001547; Bachiller, et al. Brain. Behav. Immun. (2017), doi: 10.1016/j.bbi.2016.12.018). A possible contribution by osteoclasts was also considered. However, all the serum markers of bone resorption and osteoclast activity were similar in osteoporotic and healthy postmenopausal women (data not shown). Possible osteoclast involvement was especially examined by measuring tartrate resistant phosphatase 5b (TRAP5b) in serum of an extended cohort of 99 postmenopausal women of varying BMD (FIG. 10). There was small, but insignificant inverse correlation (p=0.13, R2=0.026) between BMD and serum TRAP5b. Also, no difference was observed between healthy and patients (p=0.31, data not shown). Thus, it is unlikely that an unnoticed effect of L1 mediated action on osteoclasts can alter the present results. The higher levels of serum ALP in patients (p=0.019), although the values were within normal range, suggest a compensatory, but insufficient, bone formation to counteract the primary osteoporotic process. The functional significance of L1 reactivation in non-pathological contexts like bone development as well as early embryogenesis (Kano, et al. Genes Dev. (2009), doi: 10.1109/TLA.2016.7459581; van den Hurk, et al. Hum. Mol. Genet. (2007), doi: 10.1093/hmg/ddm108; Fadloun, Nat. Struct. Mol. Biol. (2013), doi: 10.1038/nsmb.2495; Jachowicz, et al. Nat. Genet. (2017), doi: 10.1038/ng.3945) and developing brain (Coufal, et al. Nature (2009), doi: 10.1038/nature08248; Bedrosian, et al., doi: 10.1126/science.aah3378) remains to be understood. Indeed, L1 and other transposons activity is a complex phenomenon involving several steps from long non-coding RNA (lncRNA) production, to controlled DNA damage and repair, chromatin remodeling and locus specific in cis effects at integration sites. Therefore, it is conceivable that more than one mechanism triggered by L1 reactivation would contribute to tissue specific phenotype expression. Therefore, future studies will be required to shed light whether the inhibition of L1 retrotransposons dynamics may be either a causal or a concomitant event to osteoporosis development. However, the reported facts that L1 dynamics supports osteogenesis and that L1-associated genomic structural variations distinguish healthy and osteoporotic bone in vivo, may suggest a previously unforeseen front of research for the development of strategies to mitigate bone loss in postmenopausal women and patients under antiretroviral regimen.

### II. L1 RNA Suppression Preserves H3K9M3 Heterochromatin Preventing Tissue Degeneration in Murine Progeria Model

LINE-1 (L1) elements can cause cellular toxicity by activating a proinflammatory response due to the accumulation of L1 RNA/cDNA in the cytoplasm independently of their retrotransposition. These studies investigated L1 expression in the LAKI mice to find a correlation between transcription of interspersed repetitive sequences and the onset of the ageing phenotypes.

### MATERIALS AND METHODS

### Animals and In Vivo Treatments:

All animal procedures were performed according to NIH guidelines and approved by the Committee on Animal Care at the Salk Institute. The mouse model of Hutchinson-Gilford progeria syndrome (HGPS) carrying the LMNA mutation G609G (LAKI) was generated by Carlos López-Otín at the University of Oviedo, Spain and kindly donated by Brian Kennedy at the Buck Institute.

Experiments with WT and LAKI mice were performed with mice of both genders at 8 weeks of age. For lifespan experiments, mice of both genders from a litter were randomly assigned to control and experimental groups. Any animals that appeared unhealthy before the start of experiments were excluded. No inclusion criterion was used. The mice were housed with a 12 hr light/dark cycle between 06:00 and 18:00 in a temperature-controlled room (22±1° C.) with free access to water and food.

LINE-1 specific or scramble 2′-deoxy-2′fluoro-β-d-arabinonucleotides (FANA ASO) were delivered by intraperitoneal or subcutaneous injection at the dose of 2-10 mg/Kg once every two weeks.

### Tail Tip Fibroblasts Isolation and Culture:

Tail tip fibroblasts (TTFs) were isolated from WT and LAKI mice and cultured at 37° C. in DMEM (Invitrogen) containing Gluta-MAX, non-essential amino acids, and 10% fetal bovine serum (FBS). For LINE-1 Knockdown, TTFs has been incubated with 1 μM FANA ASO dissolved in culture medium every 2 days and collected after one week for senescent marker expression or immunohistochemistry.

### Histological Analysis:

For histological analysis, tissue samples were collected at 16 weeks of age after 8 weeks of FANA-ASO injection. Mice were perfused with PBS and 10% buffered formalin solution. Subsequently, tissues were fixed overnight at 4° C. in 10% buffered formalin solution, cryopreserved overnight with 30% sucrose in PBS, embedded in OCT matrix (Kaltek) and flash frozen in liquid nitrogen. 7 μm cryosections were used for hematoxylin and eosin staining (H&E) or for immunohistochemistry.

### Immunohistochemistry:

Cells were fixed with 4% formaldehyde in PBS at room temperature (RT) for 10 min. After fixation, cells were treated with 0.5% Triton X-100 in PBS for 5 min at RT. After blocked with 4% BSA in PBS for 30 min, cells were incubated at 4° C. overnight with the primary antibody, followed by washing in PBS and incubation at RT for 1 hr with the corresponding secondary antibody. Cells were mounted using DAPI-Fluoromount-G (SouthernBiotech). Confocal image acquisition was performed using a Zeiss LSM 780 laser-scanning microscope (Carl Zeiss Jena). Images were taken at z sections of 0.25 μm intervals using the adequate lasers (488-nm, 568-nm, 633-nm and 405-nm). The laser intensity was typically set to 3%-5% transmission of the maximum intensity, and the settings were established to avoid signal saturation for any of the lasers.

Tissues sections underwent permeabilization and antigen retrieval using HistoVT One (Nacalai Tesque). Subsequently, tissue sections were blocked with 5% fraction V BSA in PBS (Sigma-Aldrich) and immunoglobulin masking reagent (Vector laboratories) and incubated overnight with primary antibody. Finally, tissue sections were incubated with secondary antibody in blocking buffer at room temperature for 60 min (invitrogen). Tissue sections were mounted with DAPI Fluoromount G mounting medium (Southern Biotech.).

### Fluorescent In Situ Hybridization:

RNA-FISH or immuno-RNA FISH in TTFs and Tissue sections was performed according to the manufacturer's standard protocol (Biosearch Technologies). Fixation was performed in 3% paraformaldehyde (PFA) for 15 min, followed by permeabilization with 1% triton X-100 for 5 minutes at room temperature prior to hybridization. Hybridization was performed at 38 degrees overnight, using 48 single-molecule probes designed to span the length of the active mouse L1spa element recognizing the majority of transcribed LINE-1 RNAs. The probe set was designed and produced by Biosearch Technologies. Custom Stellaris® FISH Probes labeled with CalFluor610 were designed against L1spa by utilizing the Stellaris® FISH Probe Designer (Biosearch Technologies, Inc., Petaluma, CA) available online at www.biosearchtech.com/stellarisdesigner.

### LINE-1 RNA In Vitro Transcription and SUV39 Enzymatic Activity Assay:

LINE-1 RNA was in vitro transcribed using MAXIscript transcription Kit (Invitrogen) using pTNC7 plasmid containing the L1spa element as a template. Before reaction pTNC7 has been linearized with NotI restriction enzyme to transcribe the full-length sense LINE-1 RNA or XhoI restriction enzyme for antisense LINE-1 RNA. Transcribed RNA was purified with RNAeasy mini kit (qiagen) following the RNA clean up protocol. Recombinant Suv39H1 (Activemotif) Histone methyltransferase (HMT) activity was assayed using EpiQuik™ Histone Methyltransferase Activity/Inhibition Assay Kit (Epigentek) following manufacturer instructions. Briefly, 1 μg of recombinant SUV39H1 was incubated with 10 ng or 50 ng of in vitro transcribed sense LINE-1 RNA. Antisense LINE-1 RNA was used as negative control as in Camacho et al. elife 2017. 1 μg of SUV39H1 alone or complexed with RNA were used for the assay in parallel with 1 μl of positive control enzyme. Absorbance was read at 450 nm on a microplate reader and HMT activity was calculated as: HMT activity=OD (sample-blank)/incubation time (Hr).

RNA Extraction and Realtime qPCR:

Total RNA was extracted from cells and tissues, using RNAeasy Plus mini kit (Qiagen) followed by cDNA synthesis using iScript Reverse Transcription Supermix for RT-PCR (Bio-Rad). qPCR was performed using SsoAdvanced SYBR Green Supermix or iQ Multiplex Powermix (Bio-Rad).

mp16-Fwd
(SEQ ID NO: 35)
CGTGAACATGTTGTTGAGGC;
mp16-Rev
(SEQ ID NO: 36)
GCAGAAGAGCTGCTACGTGA;
mp21-Fwd
(SEQ ID NO: 37)
CGGTGTCAGAGTCTAGGGGA;
mp21-Rev
(SEQ ID NO: 38)
ATCACCAGGATTGGACATGG;
mAtf3-Fwd
(SEQ ID NO: 39)
CTCTGGCCGTTCTCTGGA;
mAtf3-Rev
(SEQ ID NO: 40)
GGTCGCACTGACTTCTGAGG;
mGadd45b-Fwd
(SEQ ID NO: 41)
CGGCCAAACTGATGAATGT;
mGadd45b-Rev
(SEQ ID NO: 42)
TCTGCAGAGCGATATCATCC;
mBtg2-Fwd
(SEQ ID NO: 43)
GCGAGCAGAGACTCAAGGTT;
mBtg2-Rev
(SEQ ID NO: 44)
TAGCCAGAACCTTTGGATGG;
mMMP13-Fwd
(SEQ ID NO: 45)
TGATGAAACCTGGACAAGCA;
mMMP13-Rev
(SEQ ID NO: 46)
GGTCCTTGGAGTGATCCAGA;
mIL6-Fwd
(SEQ ID NO: 47)
TGATGCACTTGCAGAAAACA;
mIL6-Rev
(SEQ ID NO: 48)
ACCAGAGGAAATTTTCAATAGGC;
mLap2a-Fwd
(SEQ ID NO: 49)
TTCTCGAGCGACGAGGAG;
mLap2a-Rev
(SEQ ID NO: 50)
AGCCTGGGCTTATCAGTTTT;
mGapdh-Fwd
(SEQ ID NO: 51)
GGCAAATTCAACGGCACAGT;
mGapdh-Rev
(SEQ ID NO: 52)
GTCTCGCTCCTGGAAGATGG;
mL1-Fwd
(SEQ ID NO: 53)
GCGGTTCCTCAGAAAATTGG;
mL1-Rev
(SEQ ID NO: 54)
TGCCCAGGAGAGGTATTGCT;

### Senescence-Associated Beta-Galactosidase Enzymatic Activity Assay:

Senescence-associated beta-galactosidase (SA-βgal) assay was performed as described herein, briefly. Briefly, first, the cells were fixed in 4% paraformaldehyde for 5 min at room temperature. Next, the cells were washed twice with PBS and incubated overnight 37° C. in staining solution containing 40 mM citric acid/Na phosphate buffer, 5 mM K₄[Fe(CN)₆]3H₂O, 5 mM K₃[Fe(CN)₆], 150 mM sodium chloride, 2 mM magnesium chloride and 1 mg/ml X-gal. Finally, the cells were washed twice with PBS and once with methanol. The plate was dried and pictures of cells were taken using bright field microscopy.

### Results and Discussion

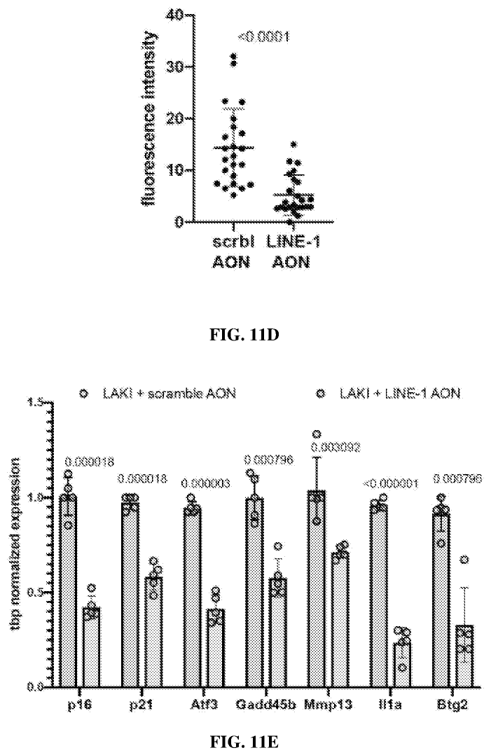

Using a multiplexed TaqMan assay, the expression of the three-active murine L1 subfamilies (L1-Tf, L1-Gf and L1-Af) was measured in tail tip fibroblasts (TTFs) isolated from wild-type (WT) and LAKI mice. In LAKI TTFs, a 3 to 6 times higher expression of L1 elements was observed (FIG. 11A). L1 expression was further confirmed using an RNA Fluorescent in situ hybridization assay (FISH) and strikingly, a strong accumulation of L1 RNA inside the nucleus was noticed (FIG. 11B). To Knock Down L1 RNA from both cytosolic and nuclear compartment L1 specific 2′F-ANA modified AON (L1-AON) was used. L1 RNA depletion was confirmed by qPCR and RNA FISH (FIG. 11C-D). Interestingly, LAKI TTFs treated with L1-AON showed a significantly lower expression of stress response genes in p53 tumor suppressor pathway (p16, p21, Atf3 and Gadd45b), senescent-associated metalloprotease Mmp13 and proinflammatory interleukin IL1a (FIG. 11E). Consistently, the number of cells positive for active senescence-associated β-galactosidase enzyme (SA-B-gal) is reduced in LAKI TTFs treated with L1-AON (FIG. 11F).

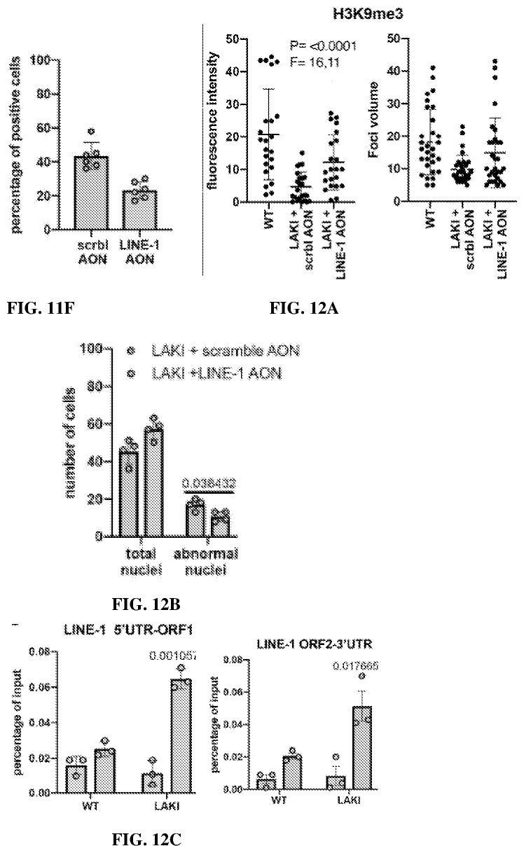

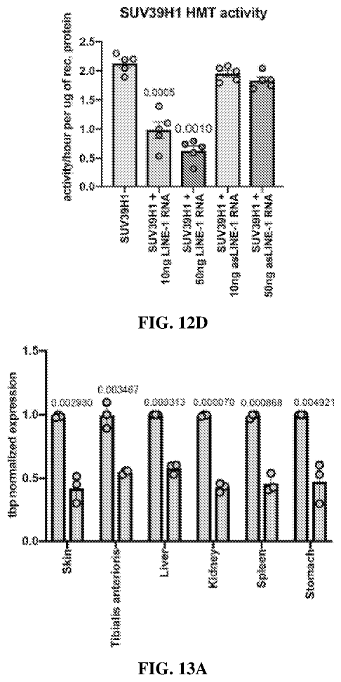

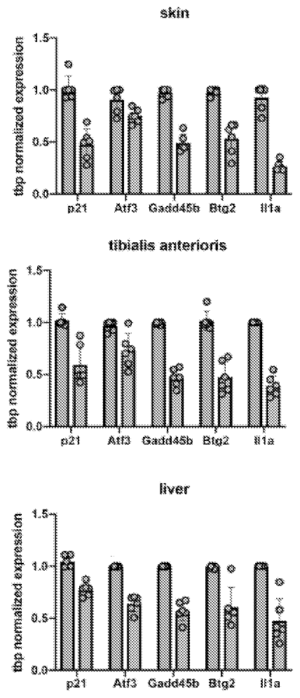

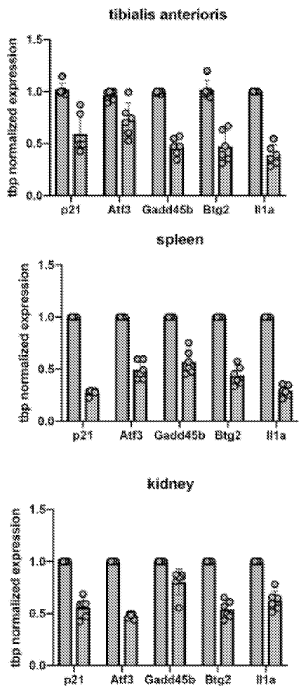

LAKI mice are characterized by significantly low levels of H3K9me3 and decondensed heterochromatin. Upon L1-AON treatment, the intensity of H3K9me3 heterochromatin foci increased in LAKI cells compared to scramble treated control cells and closer to the levels in WT (wild type) cells (data not shown, and FIG. 12A). Consequently, the number of cells with abnormal nuclei structure was also reduced (data not shown and FIG. 12B) SUV39H1/2 enzyme, the chromatin modifier responsible for the trimethylation of H3K9, is able to bind repetitive RNAs, specifically L1 RNA transcribed from the “sense” DNA strand. RNA Immuno-Precipitation (RIP) was performed and the results showed that both the 5′ end and the 3′ end of the L1 RNA is bound by SUV39H1/2 (right bar for each pair of bars) protein in LAKI TTFs (FIG. 12C). Moreover, SUV39H1/2 foci colocalized with L1 RNA spots in LAKI TTFs (data not shown). Considering that L1-ASO treatment restored the heterochromatin and reduced the expression of senescence-associated genes, further studies were conducted to determine if L1 RNA plays an inhibitory role on SUV39H1/2 accumulated in the nucleus of LAKI cells. AnH3K9 specific Histone Methyl Transferase assay was performed using a recombinant SUV39H1/2 protein in the presence of the L1 sense-oriented transcript. L1 antisense transcript was used as a negative control. L1 sense RNA exerted a strong inhibitory effect on SUV39H1/2 enzymatic activity compared to the activity of the protein alone or L1 antisense RNA (FIG. 12D).

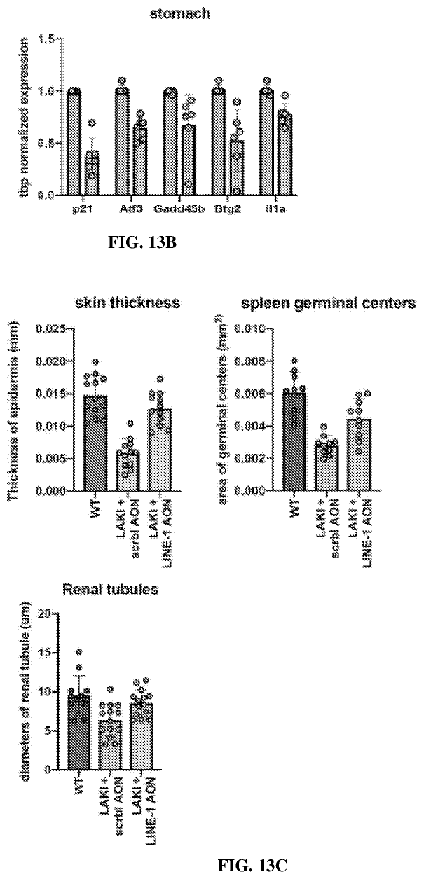

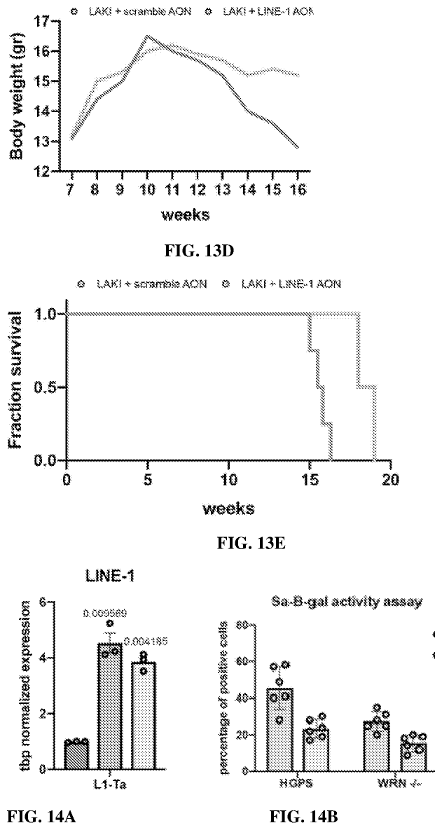

To test whether L1 RNA depletion in vivo could have any beneficial effect on LAKI mice in preventing the onset of the senescent phenotype, LAKI mice were treated with both scramble and L1-AON starting at 8 weeks of age. Mice were subjected to intraperitoneal injection of AON (T.B.D.). L1-AON treated LAKI mice were sacrificed at 16 weeks of age for molecular and histological analysis. The knockdown of L1 RNA in several tissues including skin, tibialis anterior skeletal muscle, liver, kidney, spleen and stomach was confirmed by qPCR (FIG. 13A). Importantly, 8 weeks of L1-AON treatment restored the levels of the H3K9me3 heterochromatin mark compared to scramble AON injected mice (data not shown). Moreover, L1-AON treatment reduced the expression of SASP genes in different tissues analyzed (FIG. 13B).

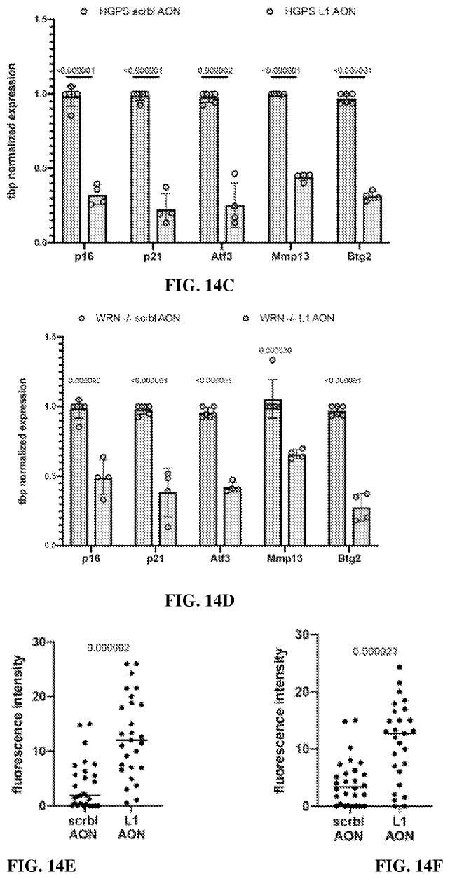

The beneficial effects of L1 FANA oligos in human cells from Progeria patients (HGPS) or recapitulating Werner Syndrome (WRN −/−) were also investigated. Consistently with data obtained in mice, both Progeria and Werner syndrome human cells are characterized by a higher expression of L1 RNA (FIG. 14A). Using human specific L1-AON cells shows a reduced SA-B-Gal activity and a reduced expression of senescent associate genes (FIG. 14B-D). Further even in the human system L1 RNA depletion is associated to the restoration of H3K9me3 heterochromatin (FIGS. 14 E-F).

To assess the efficacy of the treatment in preserving the organs from pathological changes associated with premature ageing a histological analysis of tissues that are compromised in Progeria syndrome (Cesta, 2006; Khanna et al. 1988; Kurbanand Bhawan, 1990; Zhou et al., 2008; Osorio et al., 2011) was performed. Hematoxylin-Eosin staining revealed that mice injected with L1-AON have an improved histological profile of skin, spleen, stomach and kidney (data not shown. In particular, skin is characterized by a thicker epidermal layer, germinal nuclei are wider in spleen, the volume of the epithelial layer of the stomach is higher and the diameter of the kidney glomeruli is increased (FIG. 13C). Altogether, these results confirm that a stable reduction of L1 RNA improved age-associated histological changes in multiple organs of LAKI mice.

Lastly, the bodyweight and the lifespan of treated mice was monitored. Consistently with the histological analysis, L1-AON treatment prevents the gradual loss of bodyweight typical of LAKI mice (FIG. 13D) and an increase of (15-25%) in the median lifespan was observed, compared to control and untreated mice (FIG. 13E).

Endogenous L1 elements are transcriptionally active in both physiologically (cit.) and pathologically (HGPS, FIG. 11A-11E) aged cells. This study shows that in a model of accelerated ageing like Progeria syndrome the accumulation of L1 RNA in the nucleus results in the loss of heterochromatin and increased expression of SASP related genes. Here the data show that the knockdown of this repetitive RNA using AON prevented H3K9me3 heterochromatin de-condensation and reduced the expression of age-associated genes. Furthermore, L1 RNA depletion in vivo in LAKI mice delayed the onset of the premature ageing phenotype in different tissues, loss of body weight and increased the lifespan of treated mice. Additionally, a novel function for L1 RNA as a negative regulator of SUV39H1/2 was demonstrated

In summary, in this study, for the first time shows that an antisense oligonucleotide-based therapy against a repetitive RNA is sufficient to ameliorate the ageing-associated phenotypes in LAKI mice. Therefore, AON based intervention specifically, or other interventions reducing the levels of L1 RNA in vivo, can be an attractive treatment option to devastating disease like progeria syndrome.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition for increasing L1 RNA copy number, comprising L1 RNA or a fragment thereof, in pharmaceutically acceptable carrier.

2. The composition of claim 1, wherein the L1 RNA is L1HS-Ta1.

3. The composition of claim 1, comprising the ORF1 or ORF2 of the L1 RNA.

4. The composition of claim 1, wherein the L1 RNA or fragment thereof is in an expression vector, optionally, wherein the expression vector is selected from the group consisting of plasmid, minicircle DNA (mcDNA) and viral vector.

5. (canceled)

6. The composition of claim 4, wherein the vector is selected from the group consisting of bacteriophage, baculoviruses, tobacco mosaic virus, herpes virus, cytomegalo virus, retrovirus, vaccinia virus, adenovirus and adeno-associated virus.

7. The composition of claim 4, wherein the expression vector is in a bone progenitor cell, optionally, wherein the bone progenitor cell is a bone marrow derived mesenchymal stem cell.

8. (canceled)

9. The composition of claim 7, wherein the L1 RNA comprises SEQ ID NO:1.

10. A method of increasing L1-RNA expression in a subject in need thereof, comprising administering to the subject, the composition of any one of claims in an effective amount to increase L1 RNA expression in one or more cells in the subject.

11. The method of claim 10 comprising administering bone progenitor cells genetically engineered to express L1 RNA or a functional fragment thereof to a site in the subject, in need thereof, optionally, wherein the cells are autologous cells.

12. (canceled)

13. The method of claim 11, comprising administering the cells to a site in need of bone growth or repair.

14. The method of claim 11, wherein the site is a spinal fusion site or a bone fracture site, and optionally, wherein the composition is effective increase bone mass index at a fracture site, or at a spinal fusion site in a subject diagnosed with a condition selected from the group consisting of degenerative disk disease, spondylolisthesis, spinal stenosis, scoliosis, Fractured vertebra, Infection, herniated disk and tumor.

15. (canceled)

16. The method of claim 10, wherein the composition comprises SEQ ID NO: 1.

17. A composition for reducing L1 RNA, comprising one or more agents for inhibiting L1 RNA expression, in pharmaceutically acceptable carrier.

18. The composition of claim 17, wherein the agent for inhibiting L1 RNA is selected from the group consisting of a small molecule, antisense oligonucleotide (ASO), siRNA, miRNA, shRNA, an external guide sequence and an aptamer in a pharmaceutically acceptable carrier.

19. The composition of claim 17 comprising a L1 RNA ASO, a L1 RNA ORF1 ASO and/or a L1 RNA ORF2 ASO.

20. The composition of claim 18, wherein the ASO is complementary to a fragment of L1 RNA, L1 RNA ORF1 or a L1 RNA ORF2, and optionally, wherein the ASO is no more than 24 nucleotides in length.

21. A method of reducing L1 RNA copy number in a subject in need thereof, comprising administering the composition of claim 17, to the subject.

22. The method of claim 21, wherein the composition is administered by injection.

23. The method of claim 22, comprising subcutaneously administering the composition to the subject.

24. The method of claim 17, wherein the composition alleviates one or more symptoms of subject progeria syndrome and/or skin aging.

25. (canceled)
